(19) United States
(12) Patent Application Publication
(10) Pub. No.: US 2025/0208133 A1
(43) Pub. Date: Jun. 26, 2025

(54) SENESCENT CELL SURFACE MARKERS

(71) Applicant: SENS RESEARCH FOUNDATION, Mountain View, CA (US)

(72) Inventors: Amit SHARMA, Mountain View, CA (US); Kristie KIM, Mountain View, CA (US)

(73) Assignee: SENS RESEARCH FOUNDATION, Mountain View, CA (US)

(21) Appl. No.: 18/835,014

(22) PCT Filed: Jan. 31, 2023
(86) PCT No.: PCT/US2023/061703

§ 371 Date: Jul. 31, 2024

Related U.S. Application Data
(60) Provisional application No. 63/395,289, filed on Aug. 4, 2022.

(51) Int. Cl. G01N 33/569; G01N 33/68

(52) U.S. Cl. CPC G01N 33/56966; G01N 33/6893; G01N 2333/705; G01N 2333/70596; G01N 2333/948

(57) ABSTRACT

Senescent cells are implicated in aspects of age-related decline in health and may contribute to certain diseases. Embodiments include biomarkers that are unique to senescent cells. The biomarkers have diagnostic and therapeutic uses for senescence-associated diseases and disorders. Embodiments also include methods of distinguishing non-senescent cells from senescent cells based on the presence or absence of one or more of the biomarkers. Senescent cells can be selectively labelled to detect an ailment in a subject, devise a treatment and/or determine the effectiveness of a senolytic agent. Embodiments also include methods of removing senescent cells from a patient or an affected tissue. The method can target senescent cells by inhibiting lysosomal exocytosis.

### RELATED APPLICATIONS

This is application claims priority to U.S. provisional patent application No. 63/305,227, filed on Jan. 31, 2022, and U.S. provisional patent application No. 63/395,289, filed on Aug. 4, 2022, the contents of which are incorporated herein by reference.

### FIELD OF THE INVENTION

The invention relates to diagnostics, and more specifically, it relates to diagnostic and therapeutic methods for senescence-associated diseases and disorders.

### BACKGROUND

Aging can be defined as the process of becoming older. In humans, aging represents the accumulation of changes over time and can encompass physical, psychological, and social changes. Advanced age is the greatest risk factor for many chronic diseases. More than 90% of adults aged 65 or older experience at least one chronic disease such as cancer, diabetes, or cardiovascular disease. Aging phenotypes and pathologies, including diverse age-associated diseases and disorders, are causally linked to the accumulation of senescent cell burden with age.

Senescent cells are characterized by irreversible cell-cycle arrest of proliferation-competent cells, morphological and metabolic changes, altered gene expression, chromatin reorganization, and a unique pro-inflammatory senescence-associated secretory phenotype (SASP). Senescent cells in older adults contribute to chronic inflammation and damage to surrounding tissues. It has been demonstrated that removal of senescent cells via genetic manipulation in transgenic mouse models can prevent or delay tissue dysfunction, improve age-related pathologies, and extend health span. This suggests that removal of senescent cell burden in aging adults, merits further study as a therapeutic target of interest for the treatment and prevention of the disease of aging. Senolytic drugs have also shown promising results in mice and human cell culture models.

Recent efforts have focused on methods of identifying senescent cells. The ability to distinguish senescent cells from non-senescent cells can lead to myriad therapies and diagnostics. Identification and validation of senescent cell surface markers represents an innovative and powerful advancement in the study of cellular senescence. It can reveal promising targets for selective elimination of senescent cells via immunotherapies, live image labeling, drug targeting, and differentiating non-senescent cells from senescent cells. However, the lack of a robust and universal biomarker signaling senescence and aging has impeded efforts to develop therapies. Without reliable markers, it is difficult to accurately quantify senescence burden or assess the efficacy of senotherapeutics.

Studies have identified intracellular molecules and secreted proteins that are characteristic of senescent cells. Although several proteins have been identified as upregulated on the surface of senescent cells, none have been proven to be unique to senescent cells with high certainty, particularly in vivo. For example, several surface proteins have been identified in senescent cell culture models. However, these experiments were done in cancer cell lines and senescence was induced by overexpression of p16 or p21 rather than conventional models of senescence (e.g., induction of persistent DNA damage, or oncogene activation). In another study, Kim et al. identified DDP4 as a potential marker of senescence (Kim et al., Genes Dev 31:1529-1534, 2017). However, the widespread surface expression of DPP4 in various immune cells limits its application as a biomarker in vivo. Further, elevated expression of SCAM4 or oxidized Vimentin on the cell surface of senescent cells and in the senescence-associated secretory phenotype (SASP) has been demonstrated but the surface expression of these molecules has yet not been confirmed.

Accordingly, there is a need for reliable biomarkers to identify senescent cells. The present invention includes protein biomarkers that can identify senescent cells with high specificity and reliability. Also included are methods of identifying senescent cells, diagnosing related ailments and treating age-related diseases and conditions by selective elimination of senescent cells.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

As described herein, Applicants have identified novel surface markers of senescent cells. Accordingly, embodiments include methods of identifying senescence biomarkers. Applicants identified eighteen proteins that were increased on the surface of senescent cells with both doxorubicin and irradiation treatment. Five particular proteins were further studied and validated as indicators of cellular senescence.

Embodiments also include proteins (i.e., biomarkers) identified as universal surface markers of senescent cells.

Embodiments also include the use of particular biomarkers for diagnosis, prognosis, theranosis and/or prediction of senescence-associated diseases and disorders.

Embodiments also include methods of diagnosing/identifying a senescence-associated disease or disorder using one or more biomarkers.

Embodiments also include therapies for senescence-associated diseases and disorders using senolytic agents.

Embodiments include methods of distinguishing non-senescent cells from senescent cells. The methods can include a step of comparing levels of one or more biomarkers from the non-senescent cells and senescent cells. The methods can include a step of selectively labelling senescent cells and/or selectively removing senescent cells from non-senescent cells.

Embodiments also include methods of detecting an ailment or determining a prognosis of an ailment. The methods can include steps of (a) detecting levels of one or more biomarkers in a sample from a test subject, (b) identifying the ailment or determining the prognosis of the ailment based on elevated levels of expression of the one or more biomarkers in the sample, and (c) treating the subject with a senolytic agent to prevent or ameliorate the ailment. In aspects, the ailment is a senescence-associated disease or disorder.

Embodiments also include methods of detecting an ailment in a subject. The method can include steps of (a) detecting levels of one or more biomarkers in a sample from the subject, (b) identifying an ailment based on the increased levels of the one or more biomarkers, and (c) administering a therapeutic amount of a senolytic agent based on the identity of the ailment. In aspects, the ailment is a senescence-associated disease or disorder. In aspects, the senolytic agent inhibits lysosomal exocytosis.

Embodiments also include methods of detecting or diagnosing an ailment or determining a prognosis of a subject with an ailment. The methods can include steps of (a) measuring the expression level of at least one biomarker in a test sample from the subject, (b) receiving the expression level of the at least one biomarker in the test sample by a computer, (c) comparing the expression level of the at least one biomarker in the test sample to a level in a base sample for the same at least biomarker, (d) receiving a result comparing the expression levels of the at least one biomarker in the test sample measured in a) and the base sample measured in c), and (e) diagnosing or determining the prognosis of the ailment based on altered expression of the at least one biomarker in the test sample as compared to the base sample as determined in a computer, and (f) treating the subject for the ailment based on the diagnosis or prognosis. In aspects, the ailment is a senescence-associated disease or disorder. In aspects, a senolytic agent is used for treatment. In aspects, the senolytic agent inhibits lysosomal exocytosis. The senolytic agent can be, for example, vacuolin-1 or apilimod.

In embodiments, the biomarkers include one or more of CO8A1, CATC, LYAG, IBP7, TPP1. In embodiments, the biomarkers include one or more of CO8A1, VCAM1, CATC, LYAG, PTGIS, IBP7, TPP1, CA2D1, MA2B1, CAVN2, FBLN1, LYOX, PCP, DPP4, GLCM, HEXB, PCYOX and GGALNS (as identified in Table 4). In embodiments, the biomarkers include one or more of the proteins listed on Table 2. In embodiments, the biomarkers include one or more of the proteins listed on Table 3.

In embodiments, the biomarkers include one or more of the proteins selected from TPP1, LYAG, VCAM1, GARS, CATC, NCEH1, CSPG4, ELOV1, CO8A1, IBP7, PTGIS, MA2B1, CAH2, GLCM, S10A6, DPP4, LYOX, PCP, HACD3, SDCB1, MGST3, EZRI, LEG3, FBLN1, PCYOX, HEXB, GALNS, ESYT1, ACTN4 and DHCR7 (as listed in Table 5).

In embodiments, the biomarkers include one or more of the proteins selected from LYAG, VCAM1, FBLN1, GALNS, CATC, IBP7, LRC15, CREL1, EZRI, CAVN2, NCEH1, PCYOX, SDCB1, MA2B1, TPP1, HEXB, PCP, PTGIS, CSPG4, SYNPO, CD248, FKB11, GOT1B, DPP4, CO8A1, ITA1, LYOX, GLCM, TOR1A and PHB2 (as listed in Table 6).

Embodiments also include a method of diagnosing an ailment (i.e., a senescence-associated disease or disorder) or determining a prognosis of a test subject with the ailment. The method can include steps of: a) measuring expression levels of one or more biomarkers from subjects with the ailment; b) measuring expression levels of the one or more biomarkers obtained from healthy subjects; c) comparing the expression levels of the one or more biomarkers from samples from the subjects with the ailment to the levels in samples from the healthy subjects; d) identifying biomarkers that have altered levels of expression obtained from samples from the subjects with the ailment and e) diagnosing or determining the prognosis of the ailment in a test subject by comparing of levels of biomarkers of the test subject to those from healthy subjects and subjects with the ailment. The method can include a step of treating the ailment (e.g., with a senolytic agent). The method can also include a step of creating a biomarker fingerprint from the biomarkers with altered levels of expression.

Embodiments also include a diagnostic kit for diagnosing an ailment (i.e., a senescence-associated disease or disorder). The kit can be used to identify senescent cells based on the presence of one or more biomarkers.

Embodiments also include methods of removing senescent cells for diagnostic and/or therapeutic purposes.

Embodiments also include methods for targeted delivery of senolytic or senomorphic drugs to senescent cells.

Embodiments also include methods of identifying senescent cells for targeted therapy.

Embodiments also include methods of modulating activity or modulating expression of one or more lysosomal proteins. The lysosomal proteins can be involved in lysosomal exocytosis. The one or more lysosomal proteins can be, for example, COLINA1, LOX, PBLN1, PTGIS, VCAM1, CACNA2D1, IGFBF3, GALN8, PCYOX1, SDPFR, DPP4, PRCP, CT8C, TPP1, NAN281, HEXB, CAA, NAN281 and/or GBA.

Embodiments also include methods of removing senescent cells from an affected tissue of a subject. The method can target senescent cells by inhibiting lysosomal exocytosis.

Embodiments also include a method for treating a senescence-associated disease or disorder that includes administering a senolytic agent (e.g., a small molecule), wherein the senolytic agent selectively kills senescent cells over non-senescent cells. In embodiments, the senolytic agent is Vacuolin-1 or Apilimod.

Embodiments also include a method of treating a senescence-associated disease or disorder by measuring expression levels of one or more biomarkers and administering a senolytic agent to selectively kill senescent cells. The senolytic agent can target senescent cells by inhibiting lysosomal exocytosis.

In aspects, the methods described herein can delay onset or progression of an age-related disease or condition by identifying and selectively removing senescent cells over non-senescent cells.

Embodiments include methods of using a drug conjugate for killing a senescent cell. The conjugate can include (i) a senescent cell targeting agent configured, in use, to specifically target and bind to at least one senescent cell biomarker as described herein and (ii) a cytotoxic agent, which kills the bound senescent cell. In aspects, the targeting agent is an antibody or an antigen binding fragment thereof, an aptamer, a plastic antibody or a small molecule. In aspects, the cytotoxic agent is a senolytic agent, a radioisotope, a toxin or a toxic peptide. In aspects, the senolytic is (a) an inhibitor of a Bcl-2 anti-apoptotic protein family member; (b) an MDM2 inhibitor or (c) an Akt specific inhibitor.

Embodiments include methods of using biomarkers to determine the effectiveness of a senolytic agent. In aspects, marker levels are measured in a subject before administration of the agent and after administration. The levels can then be compared. Decreased levels reflect senolytic activity.

### BRIEF DESCRIPTION OF THE DRAWINGS

The accompanying drawings illustrate aspects of the present invention. In such drawings:

FIG. 1A is a group of images of SA β-Gal-stained senescent and non-senescent IMR-90 cells. Non-senescent cells (NS) were compared with cells treated with doxorubicin (S-Doxo) and x-ray exposure (S-IR).

FIG. 1B is a graphical depiction of quantification of EdU incorporation in non-senescent (NS) and senescent (S-Doxo and IR treated) IMR-90 cells.

FIG. 1C is a graphical depiction of mRNA expression of cell cycle regulators p16 and p21 in non-senescent and senescent IMR-90 cells.

FIG. 1D is a graphical depiction of mRNA expression of cell Lamin B1 in non-senescent and senescent IMR-90 cells.

FIG. 1E is a graphical depiction of mRNA expression of SASP factors IL-6, IL-8 and IL-1a.

FIG. 1F is a group of images of Immunofluorescence assays performed to detect the expression of γ-H2AX and HMGB1 in non-senescent and senescent IMR-90 cells. Cells were stained for HMGB1 (red) and γ-H2AX (green). Nuclei were stained with Hoechst (blue). Non-senescent cells (NS) were compared with cells treated with 300 nM doxorubicin (S-Doxo) and 10 Gy of x-rays (S-IR).

FIG. 2A is a flow chart of steps in the Surfaceome-TriCEPS.

FIG. 2B is a Volcano plot that shows the abundance of cell surface proteins after cells were treated with doxorubicin.

FIG. 2C is a Volcano plot that shows the abundance of cell surface proteins after cells were treated with irradiation.

FIG. 2D is a Venn diagram of proteins that are differentially expressed upon doxorubicin treatment and irradiation compared to control cells.

FIG. 3A is a blot showing protein expression (CATC, LYAG, IBP7, TPP1, p16 INK4A and β-Actin) in non-senescent cells (NS) and senescent(S) IMR-90 fibroblasts.

FIG. 3B is a blot showing protein expression of proteins (CATC, LYAG, IBP7, TPP1, p16 INK4A and β-Actin) in non-senescent cells (NS) and senescent(S) primary endothelial cells.

FIG. 3C is a western blot comparing protein expression (CO8A1, IBP7, TPP1 and Na/K ATPase) in membrane and cytosol fractions of non-senescent cells (NS) and senescent(S) IMR-90 fibroblasts.

FIG. 3D is a western blot comparing protein expression (CATC, LYAG and Na/K ATPase) in membrane and cytosol fractions of non-senescent cells (NS) and senescent(S) IMR-90 fibroblasts.

FIG. 3E is a group of images of Immunofluorescence assays of TPP1 expression in non-senescent (NS) and senescent (Sen) IMR-90 cells.

FIG. 4A is an analysis of enriched ontology clusters among the 22 proteins identified statistically enriched terms hierarchically clustered based on Kappa-statistical similarities among their gene memberships. The 0.3 kappa score was applied as the threshold to produce term clusters.

FIG. 4B shows the results of flow cytometry analysis of LAMP-1 surface expression in NS (blue) and S (red) compared to isotype controls (gray).

FIG. 4C shows immunoblots of TFEB expression in non-senescent (NS) versus senescent(S) in IMR-90 fibroblasts.

FIG. 4D shows immunoblots of TFEB expression in NS versus S primary endothelial cells.

FIG. 4E is a graph of quantification of NS versus S fibroblasts treated with PIKfyve inhibitors vacuolin-1.

FIG. 4F is a graph of quantification of NS versus S fibroblasts treated with apilimod, from three independent experiments. Statistical analysis performed using unpaired t test comparing NS vs S cells at drug concentration of 1 μM after 48 h of treatment.

FIG. 4G shows representative light microscopy images of selective cell death of senescent cells after 48 hours of apilimod treatment (1 μM).

FIG. 4H is a bar chart of the quantification of selective cell death of senescent cells. Three fields were quantified per well (n=3) with a total of 5997 and 1293 fibroblasts counted for NS and S conditions, respectively. Error bars represent ±SEM. Statistical analysis performed using unpaired t test. * p<0.05, ** p<0.01, *** p<0.001, and **** p<0.0001

FIG. 5 shows the results of a validation study of senescent cell surface markers by flow cytometry

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “senescence” refers to gradual deterioration of functional characteristics in living organisms. Cellular senescence is often defined as a stress-induced, durable cell cycle arrest of previously replication-competent cells. The effects of senescent cells can be thought of as beneficial or detrimental with regard to host physiology and disease, although in some contexts, senescent cells affect a disease state in a complex manner both promoting and opposing certain conditions.

The term “senescence-associated disease or disorder” refers to an ailment that is associated with age and can include, for example, atherosclerosis, osteoarthritis, osteoporosis, hypertension, arthritis, cataracts, cancer, Alzheimer's disease, chronic obstructive pulmonary disease (COPD) and idiopathic pulmonary fibrosis. Other ailments (including age-related conditions) associated with age or senescence include hair graying, sarcopenia, adiposity, neurogenesis, fibrosis and glaucoma.

The term “theranosis” or “theranostics” generally refers to processes used to tailor therapy for a patient. It is the use of diagnostic tests to identify those patients who are better-suited for a drug, drugs, medicaments or therapeutics or to determine how well a drug(s), medicament(s) or therapeutic(s) is/are working.

Still other ailments associated with age or senescence include cardiovascular disease (e.g., atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke). A senescence-associated disease or disorder can also be an inflammatory or autoimmune disease or disorder (e.g., osteoarthritis, osteoporosis, oral mucositis, inflammatory bowel disease or kyphosis). A senescence-associated disease or disorder can also be a neurodegenerative disease (e.g., Alzheimer's disease, Parkinson's disease, Huntington's disease, dementia, mild cognitive impairment or motor neuron dysfunction). A senescence-associated disease or disorder can also be a metabolic disease (e.g., diabetes, diabetic ulcer, metabolic syndrome or obesity). A senescence-associated disease or disorder can also be a pulmonary disease (e.g., pulmonary fibrosis, chronic obstructive pulmonary disease, asthma, cystic fibrosis, emphysema, bronchiectasis or age-related loss of pulmonary function). A senescence-associated disease or disorder can also be an eye disease or disorder (e.g., macular degeneration, glaucoma, cataracts, presbyopia or vision loss). A senescence-associated disease or disorder is an age-related disorder that can also be renal disease, renal failure, frailty, hearing loss, muscle fatigue, skin conditions, skin wound healing, liver fibrosis, pancreatic fibrosis, oral submucosa fibrosis or sarcopenia. A senescence-associated disease or disorder can also be a dermatological disease or disorder (e.g., eczema, psoriasis, hyperpigmentation, nevi, rashes, atopic dermatitis, urticaria, diseases or disorders related to photosensitivity or photoaging).

The term “senescence-associated β-galactosidase,” “SA-β-gal” or “SABG” is a hypothetical hydrolase enzyme that catalyzes the hydrolysis of β-galactosides into monosaccharides only in senescent cells. Senescence-associated beta-galactosidase, along with p16Ink4A, can be used as a biomarker of cellular senescence.

The term “senolytic” or “senolytic agent” refers to a therapeutic such as a small molecule that can selectively or preferentially induce death of senescent cells. A senolytic agent may kill senescent cells by inducing (i.e., activating, stimulating or removing inhibition of) an apoptotic pathway that leads to cell death. Senolytic agents may be useful for treatment of senescence-associated diseases or disorders. The drugs dasatinib, quercetin, fisetin and navitoclax have potential senolytic activities.

The term “biomarker” refers generally to a DNA, RNA, protein, carbohydrate, or glycolipid-based molecular marker, the expression or presence of which in a subject's sample can be detected by standard methods (or methods disclosed herein) and is predictive or prognostic of the effective responsiveness or sensitivity of a mammalian subject with an ailment. Biomarkers may be present in a test sample but absent in a control sample, absent in a test sample but present in a control sample, or the amount of biomarker can differ between a test sample and a control sample. For example, protein biomarkers can be present in such a sample, but not in a control sample, or certain biomarkers are seropositive in the sample, but seronegative in a control sample. Also, expression of such a biomarker may be determined to be higher than that observed from a control sample. The terms “marker” and “biomarker” are used herein interchangeably.

A biomarker that is “upregulated” generally refers to an increase in the level of expression in response to a given treatment or condition. A biomarker that is “downregulated” generally refers to a decrease in the level of expression of the biomarker in response to a given treatment or condition. In some situations, the biomarker level can remain unchanged upon a given treatment or condition. A biomarker from a patient sample can be “upregulated,” i.e., the level can be increased, for example, by about 5%, about 10%, about 20%, about 30%, about 40%, about 50%, about 60%, about 70%, about 80%, about 90%, about 100%, about 200%, about 300%, about 500%, about 1,000%, about 5,000% or more compared to a reference level. Alternatively, a biomarker can be “downregulated,” i.e., the level can be decreased, for example, by about 99%, about 95%, about 90%, about 80%, about 70%, about 60%, about 50%, about 40%, about 30%, about 20%, about 10%, about 5%, about 2%, about 1% or less compared to a reference level.

The levels of expression of a biomarkers described can be assessed by any of variety of known methods for detecting expression of a transcribed nucleic acid or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In one embodiment, expression of a biomarker set is assessed using antibodies (e.g. a radio-labeled, chromophore-labeled, fluorophore-labeled, or enzyme-labeled antibody), antibody derivatives (e.g. an antibody conjugated with a substrate or with the protein or ligand of a protein-ligand pair {e.g. biotin-streptavidin}), or antibody fragments (e.g. a single-chain antibody, an isolated antibody hypervariable domain, etc.) which bind specifically with a biomarker protein or fragment thereof, including a biomarker protein which has undergone either all or a portion of post-translational modifications to which it is normally subjected in the tumor cell (e.g. glycosylation, phosphorylation, methylation etc.).

The amount of the biomarker can be measured in a test sample and compared to the “normal control level,” utilizing techniques such as reference limits, discrimination limits, or risk defining thresholds to define cutoff points and abnormal values for an ailment. The normal control level means the level of one or more biomarkers or combined biomarker indices typically found in a subject not suffering from an ailment (or prone to an ailment such as LOAD). Such normal control level and cutoff points can vary based on whether a biomarker is used alone or in a formula combining with other biomarkers into an index. Alternatively, the normal control level can be a database of biomarker patterns from previously tested subjects who did not experience the ailment over a clinically relevant time.

After selection of a set of biomarkers, well-known techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, Linear Regression or classification algorithms, Nonlinear Regression or classification algorithms, analysis of variants (ANOVA), hierarchical analysis or clustering algorithms; hierarchical algorithms using decision trees; kernel-based machine algorithms such as kernel partial least squares algorithms, kernel matching pursuit algorithms, kernel Fisher's discriminate analysis algorithms, or kernel principal components analysis algorithms, or other mathematical and statistical methods can be used to develop a formula for calculation of a risk score. A selected population of individuals is used, where historical information is available regarding the values of biomarkers in the population and their clinical outcomes. To calculate a risk score for a given individual, biomarker values are obtained from one or more samples collected from the individual and used as input data.

Tests to measure biomarkers and biomarker panels can be implemented on a variety of diagnostic test systems. Diagnostic test systems are apparatuses that typically include means for obtaining test results from biological samples. Examples of such means include modules that automate the testing (e.g., biochemical, immunological, nucleic acid detection assays). Some diagnostic test systems are designed to handle multiple biological samples and can be programmed to run the same or different tests on each sample. Diagnostic test systems typically include means for collecting, storing and/or tracking test results for each sample, usually in a data structure or database. Examples include well-known physical and electronic data storage devices (e.g., hard drives, flash memory, magnetic tape, paper printouts). It is also typical for diagnostic test systems to include means for reporting test results. Examples of reporting means include visible display, a link to a data structure or database, or a printer. The reporting means can be a data link to send test results to an external device, such as a data structure, data base, visual display, or printer.

The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., affected samples and normal or control samples). ROC curves are useful for plotting the performance of a particular feature (e.g., any of the biomarkers described herein and/or any item of additional biomedical information) in distinguishing between two populations (e.g., cases having an ailment and controls without the ailment). Typically, the feature data across the entire population (e.g., the cases and controls) are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1−specificity) of the test.

The term “detecting” or “determining” with respect to a biomarker value includes the use of both the instrument required to observe and record a signal corresponding to a biomarker value and the material(s) required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

The term “fingerprint,” “disease fingerprint,” or “biomarker signature” refers to a plurality or pattern of biomarkers that have elevated or reduced levels in a subject with disease. A fingerprint can be generated by comparing subjects with the disease to healthy subjects and used for screening/diagnosis of the disease.

The term “treating” or “treatment” refers to one or more of (1) inhibiting the disease, condition or disorder in an individual who is experiencing or displaying the pathology or symptomatology of the disease, condition or disorder (i.e., arresting further development of the pathology and/or symptomatology); and (2) ameliorating the disease condition or disorder in an individual who is experiencing or displaying the pathology or symptomatology of the disease, condition or disorder (i.e., reversing the pathology and/or symptomatology) such as decreasing the severity of disease.

The term “administration” refers to the introduction of an amount of a predetermined substance into a patient by a certain suitable method. The compositions disclosed herein may be administered via any of the common routes, as long as it is able to reach a desired tissue, for example, inhaling, intraperitoneal, intravenous, intramuscular, subcutaneous, intradermal, oral, topical, intranasal, intrapulmonary, or intrarectal administration.

The term “subject” refers to those who are susceptible to an ailment (e.g., a disease related to senescence) or who are suspected of having or diagnosed with the ailment. However, any subject to be treated with the therapeutic methods described herein is included without limitation.

The term “TriCEPS” refers to a ligand-based, receptor-capture (LRC) technique for identifying cell surface receptors and off-targets on living cells for a range of orphan ligands such as peptides and proteins. A “TriCEPS” molecule includes a chemoproteomic reagent with three moieties: a) one that binds ligands containing an amino group, b) a second that binds glycosylated receptors on living cells and c) a biotin tag for purifying the receptor peptides for identification by quantitative mass spectrometry (see, e.g., Frei et al., Nature Biotechnology 30:997-1001, 2012).

The term “antibody-dependent cellular cytotoxicity,” “ADCC” or “antibody-dependent cell-mediated cytotoxicity” refers to a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection.

The term “lysosome” refers to a membrane-bound organelle found in many animal cells. They are spherical vesicles that contain hydrolytic enzymes that can break down many kinds of biomolecules. The lumen of a lysosome has a low pH (˜4.5-5.0) that is optimal for the enzymes involved in hydrolysis. The process of hydrolytic digestion can be described in four steps. In the first step, material enters a food vacuole through the plasma membrane (i.e., endocytosis). In the next step hydrolytic enzymes are active as the food vacuole moves away from the plasma membrane. In the third step, the lysosome fuses with the food vacuole and hydrolytic enzymes enter the food vacuole. In the final step, hydrolytic enzymes digest the food particles. Lysosomes are also involved in various cell processes, including secretion, plasma membrane repair, apoptosis, cell signaling and energy metabolism.

Lysosomes act as the waste disposal system of the cell by digesting used materials in the cytoplasm, from both inside and outside the cell. Material from outside the cell is taken up through endocytosis, while material from the inside of the cell is digested through autophagy.

The term “endocytosis” or refers to a cellular process in which substances are brought into the cell. The material to be internalized is surrounded by an area of cell membrane, which then buds off inside the cell to form a vesicle containing the ingested material. The endocytic pathway of mammalian cells has distinct membrane compartments, which internalize molecules from the plasma membrane and recycle them back to the surface (as in early endosomes and recycling endosomes), or sort them to degradation (as in late endosomes and lysosomes).

The term “exocytosis” refers to a form of active transport and bulk transport in which a cell transports molecules (e.g., neurotransmitters and proteins) out of the cell. Lysosomal exocytosis is the main output pathway of the lysosome and is important for several cellular processes including plasma membrane repair, secretion and transmitter release, neurite outgrowth and particle uptake in macrophages. Upon stimulation, lysosomes translocate from the perinuclear and cytosolic regions to the plasma membrane along the microtubules. After docking, lysosomes fuse directly with the plasma membrane to release the lysosomal content into the extracellular space.

The term “lysosomal-associated membrane protein 1” or “LAMP-1” also known as lysosome-associated membrane glycoprotein 1 and CD107a (Cluster of Differentiation 107a), refers to a protein that in humans is encoded by the LAMP1 gene. The human LAMP1 gene is located on the long arm (q) of chromosome 13 at region 3, band 4 (13q34). The LAMP-1 glycoprotein is a type I transmembrane protein which is expressed at high or medium levels in at least 76 different normal tissue cell types. It resides primarily across lysosomal membranes, and functions to provide selectins with carbohydrate ligands.

The term “phosphoinositide kinase for five position containing a FYVE finger” or “PIKfyve” is a lipid kinase that phosphorylates phosphatidylinositol-3-phosphate (PI(3)P), producing PI(3,5)P2. PIKfyve plays a critical role in the endosomal and lysosomal system, regulating membrane homeostasis, endosomal trafficking and autophagy (see, e.g., Toxicol. Appl. Pharmacol. 383, 114771; 2019).

The term “apilimod” refers to an inhibitor of the lipid kinase enzyme PIKfyve.

The term “vacuolin-1” refers to a cell-permeable and water-soluble triazine based compound. Vacuolin-1 is a potent and cell-permeable lysosomal exocytosis inhibitor. Studies have demonstrated that vacuolin-1 induced rapid homotypic fusion of endosomes and lysosomes to form large and swollen structures, yet it did not disturb cell cytoskeletal network.

The term “Gene Ontology term enrichment” refers to a technique for interpreting sets of genes making use of the Gene Ontology (GO) system of classification in which genes are assigned to a set of predefined bins depending on their functional characteristics.

All numerical designations, e.g., pH, temperature, time, concentration, and molecular weight, including ranges, are to be understood as approximations in accordance with common practice in the art. When used herein, the term “about” may connote variation (+) or (−) 1%, 5%, 10%, 15% or 20% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, PA—a standard reference for various types of administration. As used herein, the term “formulation(s)” refers to a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Several types of stressors can elicit a distinct senescence phenotype that is characterized by intracellular molecules or secreted proteins. However, the surface architecture of senescent cells remains widely unknown. Applicants have identified universal surface markers uniquely present in senescent cells. Accordingly, the present invention is based on the finding that senescence can be reliably identified based on the expression of particular protein biomarkers. While the expression of biomarkers can include both up- and down-regulated levels, the biomarkers identified herein are up-regulated.

Doxorubicin and irradiation were used to induce cellular senescence in human IMR-90 fetal lung fibroblast cells. Applicants identified distinct proteins that were significantly increased on the surface of senescent cells compared to non-senescent control cells. A total of 64 statistically significant differentially abundant proteins were common to both doxorubicin-treated (S-Doxo) and irradiated (S-IR) senescent cells. Of these proteins, 22 common proteins were differentially present in both, as described below. These proteins were further validated and five proteins were short-listed based on increased protein expression by performing western blot and immunofluorescence assays. Accordingly, embodiments include proteins that are universal surface markers of senescent cells. In one embodiment, the protein biomarkers include one of more proteins referenced in Table 1. In one embodiment, the protein biomarkers include one of more proteins referenced in Table 2. In one embodiment, the protein biomarkers include one of more proteins referenced in Table 3. In one embodiment, the protein biomarkers include one of more proteins referenced in Table 4. In one embodiment, the protein biomarkers include one of more proteins referenced in Table 5. In one embodiment, the protein biomarkers include one of more proteins referenced in Table 6.

TABLE 1
Short List of Senescence Biomarkers

| | Protein | Full Name |
|---|---|---|
| 1 | CO8A1 | Collagen Type VIII Alpha 1 Chain |
| 2 | CATC | Cathepsin C |
| 3 | LYAG | Lysosomal alpha-glucosidase |
| 4 | IBP7 | Insulin-like growth factor-binding protein 7 |
| 5 | TPP1 | Tripeptidyl peptidase 1 |

Embodiments also include methods of using the biomarkers for diagnosis, prognosis and/or therapy of senescence. The methods described herein can include the combined measurement of at least one protein/peptide biomarkers and/or fragments of protein biomarkers from human serum, plasma or a derivative of blood, or blood itself.

### TriCEPS

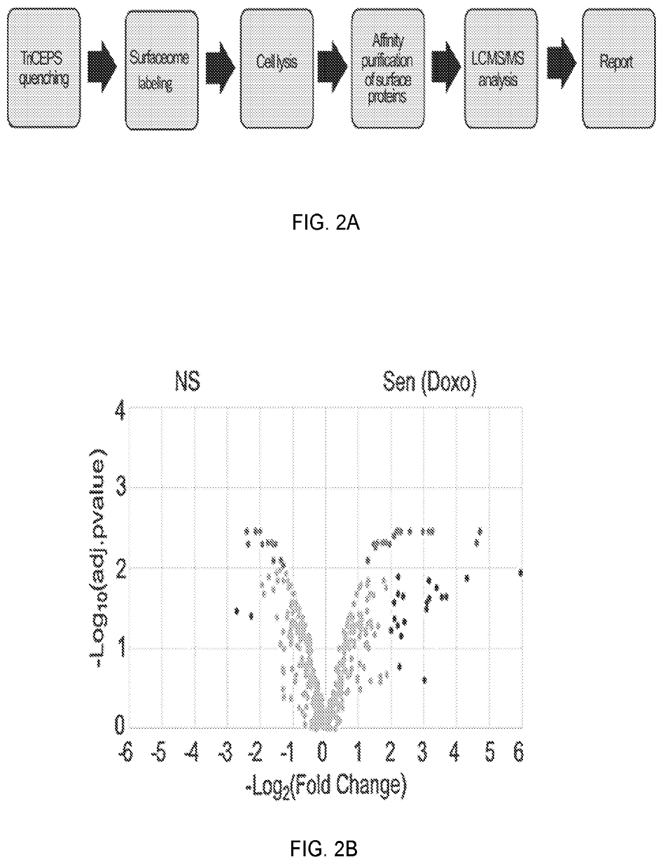

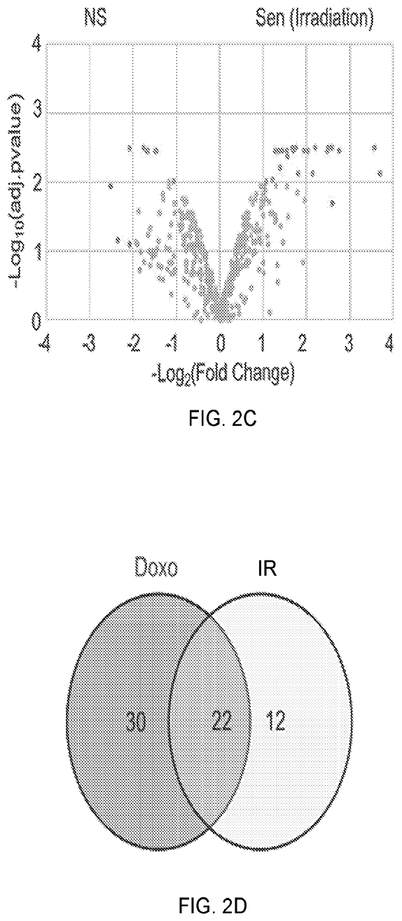

To identify novel surface makers of senescent cells, TriCEPS technology was used to capture and enrich senescent cell surface proteins. FIG. 2A is a flow chart of steps in the Surfaceome-TriCEPS method. Senescent cell surface glycoproteins were oxidized, then covalently bound to the NHS-esters of the TriCEPS v.3.0 reagent (i.e., labelling). The tagged glycoproteins were then purified, identified, and quantified by mass spectrometry-based proteomics. Table 2 lists surface proteins upregulated in cells treated with doxorubicin. Similarly, Table 3 lists surface proteins upregulated in cells treated with irradiation. The proteins common to both treatments (i.e., found in both doxorubicin and irradiation treatment) are identified in Table 4.

FIG. 2B is a series of volcano plots depicting the abundance of roughly 50 (B-i) surface proteins upregulated by doxorubicin treatment when compared to non-senescent cells and 35 (FIG. 2C) surface proteins upregulated by irradiation. Proteins with an adjusted p-value of less than 0.01 were defined as “high confidence” proteins. Proteins with statistical significance less than 0.01, but with a fold change difference greater than 4 were defined to be “medium confidence” differentially expressed. Table 5 lists surface proteins identified from cells treated with doxorubicin-treated (S-Doxo) that had adjusted p-values of less than 0.01. Table 6 lists surface proteins identified from cells treated with irradiation (S-IR) that had adjusted p-values of less than 0.01.

FIG. 2D is a Venn diagram of proteins discovered to be differentially abundant upon doxorubicin treatment and irradiation compared to control cells. To investigate differentially abundant proteins that were common to both doxorubicin-treated and irradiated senescent cells, 64 statistically significant differentially abundant proteins were analyzed. 22 common proteins were identified that were differentially present in cells treated with doxorubicin treatment and irradiation.

There are many important advantages of identification of novel surface markers of senescent cells including:
1. Biomarker of senescent cells;
2. Cleanup of senescent cells from cell therapy products and tissue engineering products ex vivo;
3. Removal of senescent cells for therapeutic purposes;
4. Removal of senescent cells for diagnostic purposes;
5. Development of a targeted dye for senescent cells using the senescent cell surface marker;
6. Targeted delivery of senolytics or senomorphic drugs or other drugs or therapeutics;
7. Enhancing the cytotoxicity of cells towards senescent cells (for example via antibody-dependent cellular cytotoxicity or ADCC);
8. Development of immunotherapeutic approaches to inhibit to kill senescent cells by CAR-modified T cells, NK cells, or γδ T cells, macrophages, dendritic cells;

### Lysosomal Proteins

Published literature has shown evidence that organelles delivering cell material to lysosomes for degradation (e.g., autophagosomes and endosomes) can change their destination from fusion with lysosomes to fusion with the plasma membrane for extracellular release by a phenomenon known as lysosomal exocytosis (see, e.g., Buratta et al., Int J Mol Sci. 2020 Apr. 8; 21(7):2576). Lysosomal exocytosis is also known to play a crucial role in plasma membrane repair in all cell types (see, e.g., Samie et al., J Lipid Res. 2014 June; 55(6):995-1009). This process helps to restore plasma membrane integrity after injury, as lysosomes located near the wounded site quickly migrate and fuse with the plasma membrane, efficiently resealing the damage and remodeling the tissue.

Applicants identified lysosomal proteins that were increased on the surface of senescent cells compared to non-senescent control cells. The proteins are identified in the figures. Accordingly, embodiments include methods of modulating the activity of one or more lysosomal proteins to treat a senescence-associated disease or disorder.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof

### Methods

### Cell Culture

IMR-90 fibroblasts (ATCC, USA: Cat #CCL-186) were maintained at 37° C. in humidified air containing 5% CO₂ and 3% O₂. Fibroblasts were used at population doubling level (PDL) 30-47 and maintained in DMEM complete media containing Dulbecco's Modified Eagle's Medium (DMEM) (Corning; Cat #10-013-CV) supplemented with 10% Fetal Bovine Serum (FBS) (Millipore Sigma, USA; Cat #F4135) and 1×Penicillin-Streptomycin (Corning; Cat #30-001-CI). Cumulative PDL was calculated using the following equation:

$$PDL = \frac{log H - log S}{log 2}$$

where H is the number of cells at harvest and S is the number of cells seeded. Primary human endothelial cells purchased from Coriell Institute for medical research (AG10770) were maintained in promo cell basal medium MV2 (PromoCell; Cat #C-22221) supplemented with Growth Medium MV 2 Supplement Pack (PromoCell; Cat #C-39221) and assayed within less than a passage number of 10. Endothelial cells were maintained at 37° C. in humidified air containing 5% CO₂. Quiescence was induced by replacing culture media with media containing 0.2% FBS for 24-72 h before analysis. All cells were mycoplasma free.

### Senescence Induction.

Human IMR-90 fibroblasts (ATCC, USA) were treated with 300 nM of doxorubicin hydrochloride (Millipore Sigma, USA; Cat #504042) in DMEM complete media for 24 hours or ionizing radiation (10 Gy X-ray) and maintained in culture. Primary human endothelial cells were treated with 250 nM of doxorubicin in promo cell basal medium supplemented with Growth Medium MV 2 Supplement Pack for 24 hours and maintained in culture. Senescent cells were used 7-13 days following treatment.

### Senescence-Associated β-Galactosidase Staining

Senescence burden in IMR-90 cells was determined by performing a senescence-associated β-galactosidase (SA-β-gal) activity assay as has been previously reported (Laberge et al. 2015), using the Senescence Detection Kit (BioVision; Cat #K320), following the manufacturer's instructions. For the assay, fibroblasts were plated (8×10⁴ per well) one day before senescence induction in a 6-well cell culture plate (Greiner Bio-One; Cat #657160) with 2.5 ml of DMEM complete media per well. Non-senescent cells were plated (5×10⁴ per well) also in six-well cell culture plates four days before SA-β-gal staining. Media was replaced with low-serum media 24 hours before staining. Staining was performed 13 days after doxorubicin treatment of IMR-90 cells. During staining, cells were incubated for 24 hours at 37° C. in the absence of CO₂, then visualized by bright-field microscopy and imaged.

### Real-Time Quantitative PCR

Non-senescent fibroblasts were plated (1.5×10⁴/well) in T-25 cell culture flasks (Cellstar; Cat #690160) with four ml/flask of DMEM complete media four days before collecting cells. For senescent cells, fibroblasts were plated (2.5×10⁴/well) one day before senescence induction, also in T-25 cell culture flasks. Senescent cell pellets were collected 14 days after doxorubicin treatment or irradiation. Media was replaced with low-serum media 24 hours before all cell pellets were collected. Cell pellets were stored at −80° C. before RNA isolation. The total RNA was isolated from cell pellets using Quick-RNA MiniPrep (Zymo Research; Cat #R1055), following the manufacturer's protocol. 1 μg of total RNA per sample was reverse transcribed using PrimeScript RT Master Mix (Takara; Cat #RR036B), and cDNA was analyzed by real-time qPCR using TaqMan Fast Advanced Master Mix (Applied Biosystems; Cat #4444557) (StepOnePlus™ Real-Time PCR System). Gene expression analyses were performed with Applied Biosystems TaqMan Gene Expression single-tube assays. All reactions were performed in triplicate, and relative expression levels of each gene were normalized to actin. The relative expression of mRNA was determined using the comparative threshold (Ct) method by normalizing target cDNA Ct values to that of actin.

### Cell Proliferation Analysis.

Cell proliferation was measured via incorporation of EdU in dividing cells using Click-IT EdU Cell Proliferation Kit for Imaging, Alexa Fluor 488 dye (Thermo Scientific; Cat #C10337), following the manufacturer's instructions. Non-senescent fibroblasts were plated (2×10⁴/well) in Nunc Lab-Tek 8-well Chamber Slides (Thermo Scientific; Cat #177402PK) in 0.5 ml/well DMEM complete media 1 day before staining. For senescent cells, fibroblasts were plated (1×10⁴/well) 1 day before induction of senescence. Staining was performed seven days after senescence induction. EdU incorporation was visualized and imaged using a fluorescence microscope. Percent of EdU positive cells was quantified by counting total number of cells and EdU positive cells over 12 microscopic fields per assay.

### Immunofluorescence (IF)

Fibroblasts were plated for senescence induction (1-2×10⁴/well) one day before doxorubicin treatment in a black 96-well plate with square wells and flat, clear bottom (Ibidi; Cat #89626), with 250 μl/well of DMEM complete media. Non-senescent cells were plated (1-2×10⁵/well) 1 day before staining. All staining for immunofluorescence was performed 7 days after senescence induction of IMR-90 cells. Cells were fixed with 200 μl/well of 4% Paraformaldehyde in 1×PBS (Thermo Scientific; Cat #AAJ19943K2) for 15 minutes at room temperature, carefully rinsed with 1×PBS (Corning; Cat #21-031-CV), then permeabilized with 300 μl per well of 0.5% Triton X-100 for 10 minutes at room temperature. Cells were then rinsed once with 1×PBS and incubated with 250 μl per well of anti-γH2AX [p Ser139] (Novus Biologicals; Cat #NB100-74435) and anti-HMGB1 (abcam; Cat #ab18256) antibodies diluted in 5% BSA (Research Products International; Cat #A30075) in 1×PBS overnight at 4° C. Subsequently, cells were washed 5 times with 1×PBS and incubated with 250 μl/well of Alexa Fluor 488 goat anti-mouse antibody (Invitrogen; Cat #A11029), Alexa Fluor 546 goat anti-rabbit antibody (Invitrogen; Cat #A11010), and Hoechst 33342, trihydrochloride, trihydrate (Invitrogen; Cat #H3570) diluted in 5% BSA for 20 min at room temperature in the dark. Cells were washed 5 times with 1×PBS, then 200 μl of 1×PBS was added to each well before images were acquired with Molecular Devices Image Express Micro (Molecular Devices, San Jose, CA, USA). Seven images were acquired per well, with six wells captured per condition (NS or S).

### Immunoblotting and Isolation of Cell Membrane

For analysis of whole cell lysate, cells were lysed in RIPA buffer (20 mM Tris-HCl PH 7.5, 150 mM NaCl, 1 mM Na₂ EDTA, 1 mM EGTA, 1% NP-40, 1% sodium deoxycholate 2.5 mM sodium pyrophosphate, 1 mM beta-glycerophosphate, 1 mM Na₃VO₄, and 1 μg/ml leupeptin) containing 1×protease inhibitor cocktail (Cell Signaling Technology; Cat #5871). Cell suspensions were incubated for 10 minutes on ice, followed by microcentrifugation at 4° C. for 15 minutes to clear the lysate of cell debris. To extract cell membrane proteins, cell lysates were prepared using the Mem-PER Plus Membrane Protein Extraction Kit (Thermo Scientific; Cat #89842). Protein concentration was quantified using the BCA Protein Assay (Thermo Scientific; Cat #23227). Equal amounts of protein were separated by SDS/PAGE and transferred onto PVDF membranes using a semi-dry transfer apparatus (Bio-Rad). Membranes were incubated with primary antibody overnight at 4° C., followed by incubation with secondary antibody for one hour at room temperature. Blots were developed using Pierce ECL Western Blotting substrate (Thermo Scientific; Cat #32209). The following antibodies were used for immunoblotting analysis: COL9A1 (Sigmal Aldrich, USA; HPA053107), Cathepsin C (D-6) (Santa Cruz; sc-74590), GAA (Sigma-Aldrich, USA; HPA029126), IGFBP7 (Abcam; ab171085), TPP1 clone 2E12 (Millipore Sigma, US; MABN1806), and Na/K ATPase (Invitrogen; MA5-32184). Other primary antibodies that were used include anti-TFEB (Santa Cruz Biotechnology; Cat #sc-166736), anti-p16 (Santa Cruz; Cat #56330) and anti-β Actin (Cell Signaling; Cat #4967). The following secondary antibodies were included: anti-rabbit IgG H&L (HRP) (Abcam; Cat #ab6802) and anti-mouse IgG H&L (HRP) (Thermo Scientific; Cat #G-21040).

### TriCEPS™-Based Ligand-Receptor Capture (LRC-TriCEPS).

Dualsystems Biotech AG, TriCEPS™ V.3.0 for capturing the proteins at the cell surface of living cells kit (Cat. Number: P0529) was used to identify surface proteins enriched in IMR-90 cells made senescent by treatment with doxorubicin or exposure to ionizing radiation as described before using the manufacturers' instructions. Three separate 50 mL tubes for NS, Sen (IR) and Sen (Doxo) each containing 24×10⁶ IMR-90 cells were scraped into PBS (pH 6.5) to detach the cells. Subsequently, cells were washed and cooled to 4° C. The remaining steps were all performed at 4° C. Sodium metaperiodate (1.5 mM) was added to the cell suspension for mild oxidation of cells surface proteins and cells were incubated at 4° C. in the dark for 15 min under gentle rotation. The cells were then washed twice at 300×g for 5 minutes, then resuspended in 20 ml of Surfaceome buffer. For Surfaceome labeling, quenched TriCEPS v.3.0 was added to the oxidized cells and incubated in the dark for 90 minutes on a rotator under gentle agitation. The samples were centrifuged at 1200 g for 5 minutes at 4° C. Cell pellets were stored at −80° C. before being sent to Dualsystems, where samples were purified using solid phase chromatography, stringently washed to remove unspecific interactions, reduced, alkylated and digested with trypsin. The tryptic peptides were then collected for LC-MS/MS analysis. To allow for statistical analysis, the experiment was performed in biochemical triplicates.

### Mass Spectrometry

Surfaceome-TriCEPS samples were analyzed on a Thermo Orbitrap Elite spectrometer fitted with an electrospray ion source in triplicates. Tryptic peptides were measured in data dependent acquisition mode (TOP20) in an 80-minute gradient using a 15 cm C18 packed column.

### Data Analysis

PROGENESIS® software was used for raw file alignment and feature detection. Comet search engine was used for spectra identification. The Trans proteomic pipeline was used for statistical validation of putative identifications and protein inference. Upon protein inference, relative quantification of samples was performed based on ion extracted intensity, and differential protein abundance was tested using a statistical ANOVA model followed by multiple testing correction. This model assumes that the measurement error follows Gaussian distribution and views individual features as replicates of a protein's abundance and explicitly accounts for this redundancy. It tests each protein for differential abundance in all pairwise comparisons and reports the p-values. Next, p-values are adjusted for multiple comparisons to control the experiment-wide false discovery rate (FDR). The adjusted p-value (qvalue) obtained for every protein is plotted against the magnitude of the fold enrichment between the two experimental conditions.

The quantitative differences between perturbations are shown as a volcano plot. A volcano plot combines a measure of statistical significance from a statistical test (in this case adjusted p-value from an ANOVA model) with the magnitude of the change, enabling quick visual identification of proteins that display changes that are also statistically significant. The x-axis represents the mean ratio fold change (on a log 2 scale). The y-axis represents the statistical significance p-value of the ratio fold change for each protein (on a −log 10 scale). Proteins that are differentially abundant in one of the samples, will plot either left or right of the x-axis origin, indicating in which sample that protein is enriched.

### Flow Cytometry

Cells were resuspended in 100 μl of PBS. Cells were then incubated with APC-conjugated anti-human CD3 antibody (Miltenyi Biotec; Cat #130-113-135). LAMP-1 antibody (BioLegend; Cat #328601) was incubated with the cells on ice for 30 minutes. Cells were washed with 1 ml of ice cold PBS and resuspended in 100 μl of ice cold PBS. APC-conjugated anti-mouse IgG (H+L) secondary antibody (Thermo Scientific; Cat #A-865) was incubated with the cells for 30 min on ice. Then, cells were washed with 1 ml of cold PBS and resuspended in 100 μl of ice cold PBS. Data were collected by flow cytometer (DB Accuri C6). Cell viability was determined by PI staining and live cells were gated for downstream analysis. Data were analyzed using Flowlogic software (Miltenyi Biotech, Germany).

### Crystal Violet Staining

Cell viability after 48 h of apilimod (1 μM) or vacuolin-1 (1 μM) treatment was estimated by crystal violet. Cells in 6-well cell culture plates (Greiner Bio-One; Cat #657160) were washed twice with PBS, incubated with 4% paraformaldehyde for 15 minutes at room temperature, then stained with 0.25% crystal violet for 10 minutes. Cells were washed with deionized water and imaged, 3 images per well.

### Impedance Measurements

For measurement of background values, 50 μl of complete medium was added to E-Plates 96 (Agilent). Cells were seeded in a total of 200 μl of medium per well at a density of 10,000 cells per well. Cell attachment was monitored using the RTCA MP (Agilent) instrument and the RTCA software (Agilent). Media was replaced with media containing either DMSO, vacuolin-1 or apilimod. Cells treated with 0.2% Triton X-100 was used as a 100% dead cell positive control for cytotoxicity assays. After drug addition, impedance measurements were recorded every 15 minutes. All experiments were performed in at least 5 replicates for each dose. Changes in impedance were expressed as a cell index (CI) value, which derives from relative impedance changes corresponding to cellular coverage of the electrode sensors, normalized to baseline impedance values with medium only. To analyze the acquired data, CI values were exported, and percentage of attached cell surface was calculated in relation to positive controls and DMSO-treated cells.

Methods of Diagnoses using Biomarkers

One or more of the biomarkers can be used in a method of diagnosing an ailment (e.g., a senescence-associated disease or disorder) or determining a prognosis of a test subject with an ailment. In this manner, one biomarker or a combination of 2, 3, 4, 5, 6, 7, 8, 9, 10, 11, 12, 13, 14, 15, 16, 17, 18, 19, 20, 21 and/or 22 biomarkers can be used in a method of diagnosing an ailment or determining a prognosis of a test subject with an ailment. In this manner, at least one biomarker or a combination of at least 2, 3, 4, 5, 6, 7, 8, 9, 10, 11, 12, 13, 14, 15, 16, 17, 18, 19, 20, 21 and/or 22 biomarkers can be used in a method of diagnosing an ailment or determining a prognosis of a test subject with an ailment.

In this manner, no more than one biomarker or a combination of no more than 2, 3, 4, 5, 6, 7, 8, 9, 10, 11, 12, 13, 14, 15, 16, 17, 18, 19, 20, 21 and/or 22 biomarkers are used in a method of diagnosing an ailment or determining a prognosis of a test subject with an ailment. In this manner, about one biomarker or a combination of about 2, 3, 4, 5, 6, 7, 8, 9, 10, 11, 12, 13, 14, 15, 16, 17, 18, 19, 20, 21 and/or 22 biomarkers are used in a method of diagnosing an ailment or determining a prognosis of a test subject with an ailment.

In a first step of a method, the expression levels of one or more proteins are measured in plasma samples from subjects with an ailment. In an embodiment, the expression levels of one biomarker or a combination of 2, 3, 4, 5, 6, 7, 8, 9, 10, 11, 12, 13, 14, 15, 16, 17, 18, 19, 20, 21 and/or 22 biomarkers are used to generate a footprint or signature for subsequent diagnosis of patients. In an embodiment, the expression levels of at least one biomarker or a combination of at least 2, 3, 4, 5, 6, 7, 8, 9, 10, 11, 12, 13, 14, 15, 16, 17, 18, 19, 20, 21 and/or 22 biomarkers are used to generate a footprint or signature for subsequent diagnosis of patients.

In an embodiment, the expression levels of no more than one biomarker or a combination of no more than 2, 3, 4, 5, 6, 7, 8, 9, 10, 11, 12, 13, 14, 15, 16, 17, 18, 19, 20, 21 and/or 22 biomarkers are used to generate a footprint or signature for subsequent diagnosis of patients. In an embodiment, the expression levels of about one biomarker or a combination of about 2, 3, 4, 5, 6, 7, 8, 9, 10, 11, 12, 13, 14, 15, 16, 17, 18, 19, 20, 21 and/or 22 biomarkers are used to generate a footprint or signature for subsequent diagnosis of patients.

Next, expression levels of the same proteins are measured in plasma, blood or tissue samples from healthy subjects. This is used as a control. Thereafter, samples from healthy patients can be compared to identifying proteins that have altered levels of expression in the plasma samples from the subjects with an ailment. A biomarker fingerprint or signature can be created from the proteins with altered levels of expression. This can be used for diagnosing or determining the prognosis of an ailment in the test subject by comparing of levels of proteins from plasma of the test subject. Conventional statistical analysis can be used to determine, for example, confidence levels.

### Diagnostic Kit for Screening an Ailment

The following working example is based on configurations described above. Embodiments of the invention can be compiled into a diagnostic kit for diagnosing an ailment such as a senescence-associated disease or disorder. The kit can identify one or more target cells that have the biomarkers for ailment in plasma from a test subject.

The kit can include reagents that can be used to identify variations in expression levels of one or more proteins in a sample from a test subject. The expression levels of the proteins can be used in a comparison/analysis of test samples with a fingerprint indicative of the presence of an ailment.

In certain embodiments, the present disclosure provides kits for diagnosing an ailment related to senescence. The kits can include one biomarker or a combination of 2, 3, 4, 5, 6, 7, 8, 9, 10, 11, 12, 13, 14, 15, 16, 17, 18, 19, 20, 21 and/or 22 biomarkers disclosed herein. The skilled artisan will appreciate that the number of biomarkers may be varied without departing from the nature of the present disclosure, and thus other combinations of biomarkers are also encompassed by the present disclosure. The skilled artisan will know which one biomarker or a combination of 2, 3, 4, 5, 6, 7, 8, 9, 10, 11, 12, 13, 14, 15, 16, 17, 18, 19, 20, 21 and/or 22 biomarkers to use based on the symptoms of the patient suffering from an ailment.

In a specific embodiment, a kit includes the one biomarker or a combination of 2, 3, 4, 5, 6, 7, 8, 9, 10, 11, 12, 13, 14, 15, 16, 17, 18, 19, 20, 21 and/or 22 biomarkers disclosed herein. The kit can further optionally include instructions for use. The kit can further optionally include (e.g., comprise, consist essentially of, consist of) tubes, applicators, vials or other storage containers with the above-mentioned biomarker and/or vials containing one or more of the biomarkers. In an embodiment, each biomarker is in its own tube, applicator, vial or storage container or 2, 3, 4, 5, 6, 7, 8, 9, 10, 11, 12, 13, 14, 15, 16, 17, 18, 19, 20, 21 and/or 22 biomarkers are in a tube, applicator, vial or storage container.

The kits, regardless of type, will generally include one or more containers into which the one biomarker or a combination of 2, 3, 4, 5, 6, 7, 8, 9, 10, 11, 12, 13, 14, 15, 16, 17, 18, 19, 20, 21 and/or 22 biomarkers are placed and, preferably, suitably aliquoted. The components of the kits may be packaged either in aqueous media or in lyophilized form.

### Example 1

### Genotoxic Stress-Induced Model of Senescence

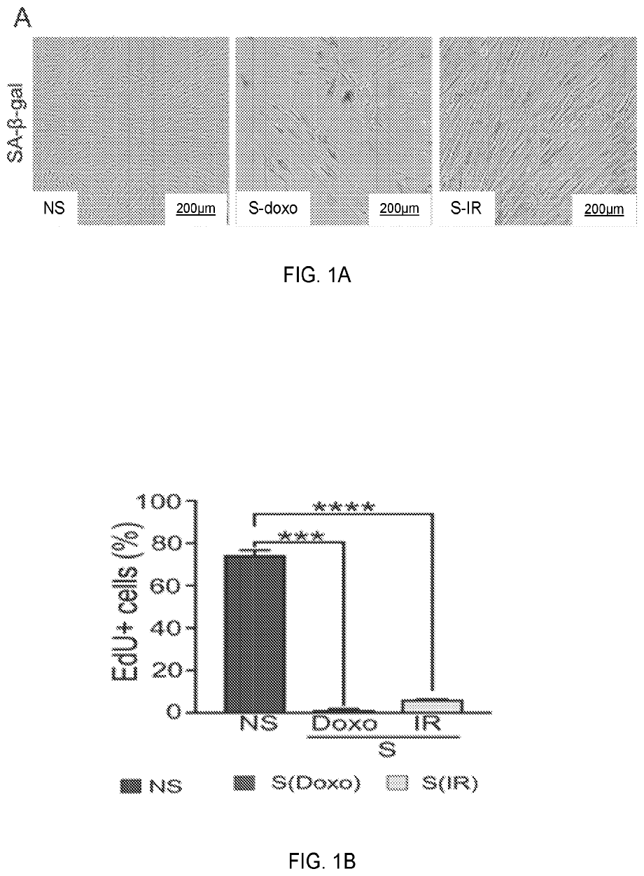

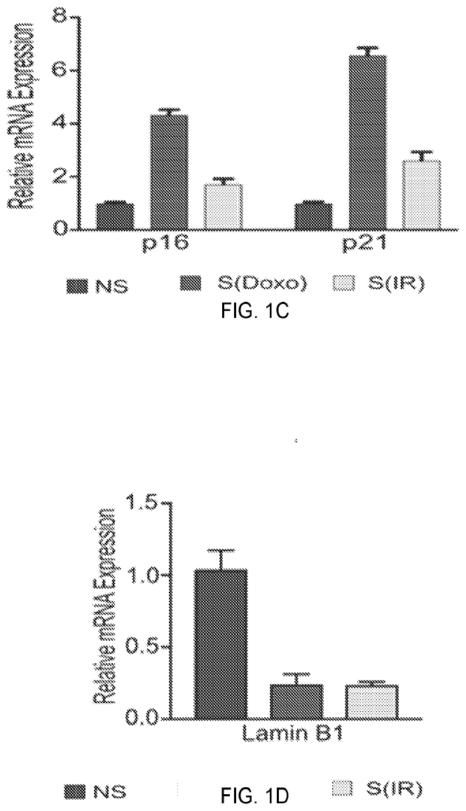

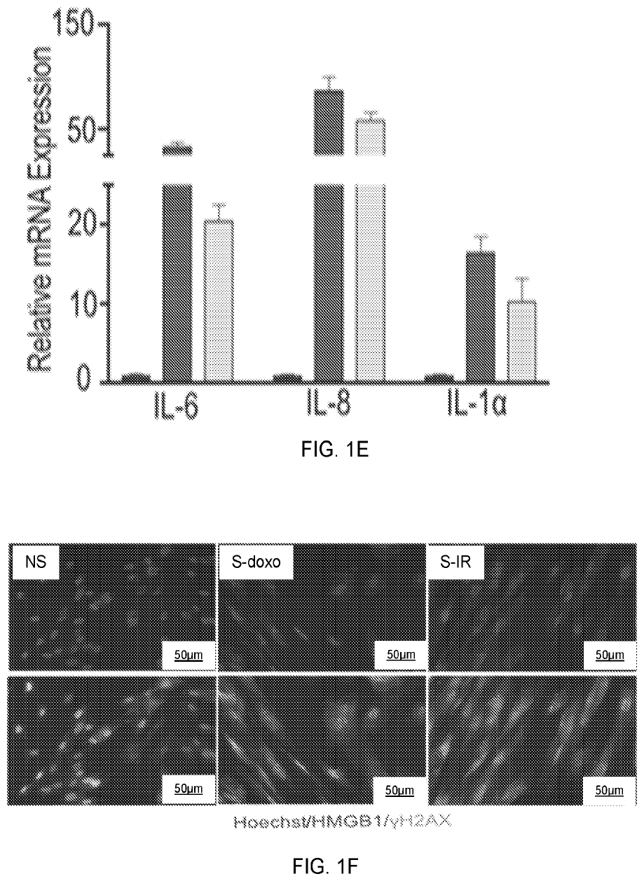

To identify novel senescent cell surface markers, IMR-90 human fetal lung fibroblast cells were used, which are commonly used in cell culture models of senescence. Senescence burden in IMR-90 cells was determined by observing senescence-associated β-galactosidase (SA-β-gal) activity, which can be used to measure the increased activity of lysosomal beta-galactosidase that is characteristic of senescent cells (see. e.g., Itahana et al., Methods Mol Biol 371:21-31, 2007). A robust induction of lysosomal beta-galactosidase was observed in IMR-90 cells treated with either doxorubicin (300 nM) or 10 Gy of x-rays when compared to quiescent cells 13 days after treatment as shown graphically in FIG. 1A.

Another characteristic of senescent cells is the loss of cell proliferation. A significant decline in the numbers of proliferating cells was observed seven days after treatment with doxorubicin or irradiation, as measured by EdU cell proliferation assay as shown in FIG. 1B. In both doxorubicin-treated (S-Doxo) and irradiated (S-IR) IMR-90 cells, non-senescent control cells exhibited greater proliferation activity than senescent cells.

Cells were plated in 8-well chamber slides and counted 7 days post-treatment. Four fields were quantified per well (n=3) with a total of 1276, 672, and 1102 cells counted for NS, S(Doxo), and S(IR) cells, respectively.

Loss of proliferation was further confirmed by measuring the increased expression of cell cycle checkpoint markers such as p16INK4A and p21CIP1A in doxorubicin-treated and irradiated fibroblasts compared to non-senescent fibroblasts (see, e.g., Baker et al., Nature 530:184-189, 2016). The results are graphically depicted in FIG. 1C.

Because senescent cells undergo significant morphological changes including changes in nuclear lamina such as decreased expression of Lamin B1, Applicants measured mRNA expression of nuclear Lamin B1. The results demonstrate a significant reduction in the expression of Lamin B1 in both doxorubicin-treated and irradiated senescent IMR-90 cells compared to quiescent control cells as shown in FIG. 1D. Chromatin changes caused by loss of Lamin B1 lead to increased expression of p53 and p38 MAPK, which are causally linked to a robust increase in SASP expression (see, e.g., Freund et al., Mol Biol Cell 23:2066-2075, 2012; Lopes-Paciencia et al., Cytokine 117:15-22, 2019). Applicants demonstrated that the expression of several prototypical SASP factors and observed a robust induction of IL-6, IL-8 and IL-1a by qRT-PCR analysis as shown in FIG. 1E. Further, previous studies have shown that senescent cells exhibit loss of non-histone, nuclear DNA binding protein, HMGB1 (Davalos et al., J Cell Biol 201:613-629, 2013). Applicants confirmed nuclear loss of HMGB1 from both doxorubicin-treated and irradiated senescent cells as shown in FIG. 1F. Further, Applicants demonstrated persistent DNA damage foci in senescent cells as measured by gH2AX staining in senescent cells compared to quiescent cells as shown in FIG. 1F. Collectively, these data demonstrate that the genotoxic stress-induced models of senescence successfully induced a robust senescence phenotype in IMR-90 fibroblasts.

### Example 2

### Identification of Senescent Cell Surface Markers

Cellular responses are generally mediated through interactions with proteins located on the cell surface. Progress has been made in elucidating the molecular mechanisms involved in cellular senescence. However, surface characterization of senescent cells is lacking. Several reports suggest identification of senescent cell surface makers. However, further analysis has shown that these markers are not universal and their surface expression is not unique to senescent cells (see, e.g., Frescas et al., Proc Natl Acad Sci USA 114: E1668-E1677, 2017).

### Example 3

### Surfaceome Characterization of Senescent IMR-90 Fibroblasts

Following the Surfaceome-TriCEPS experiment, roughly 50 surface proteins were identified to be differentially abundant in senescent cells (S-Doxo) when compared to non-senescent (NS) cells using protocols described above. Whereas the abundance of roughly 35 surface proteins were affected in senescent cells following irradiation. The differentially abundant proteins were assigned to two groups: high and medium confidence. High confidence differentially expressed are defined as proteins with an adjusted p-value of less than 0.01 (−Log 10 (adj. p value)>2). Medium confidence differentially expressed are defined as proteins with a fold change difference greater than 4, but with a statistical significance of less than 0.01. These results are presented in Volcano plots (FIG. 2B, FIG. 2C) depicting the comparison between NS and senescent cells (S-Doxo or S-IR).

Table 2 is a list of surface proteins identified from cells treated with doxorubicin-treated (S-Doxo) that had adjusted p-values of less than 0.01. Table 3 is a list of surface proteins identified from cells treated with irradiation (S-IR) that had adjusted p-values of less than 0.01. To investigate differentially abundant proteins that were common to both doxorubicin-treated and irradiated senescent cells, a total of 64 statistically significant differentially abundant proteins were analyzed. 22 common proteins were identified that were differentially present in both, as shown in FIG. 2B and FIG. 2C.

From the 22 proteins that were common between the two perturbations, Applicants focused on the 18 proteins that were upregulated in both conditions (S-Doxo, S-IR) compared to NS cells. High confidence candidates were chosen that were at least 2.2 log 2 fold higher in senescent cells compared to NS controls. The list was further narrowed by selecting those that were not expressed on the surface of vital tissue samples according to the protein atlas (see. e.g., www.proteinatlas.org). In this way, the following five proteins were short-listed CO8A1, CATC, LYAG, IBP7, and TPP1.

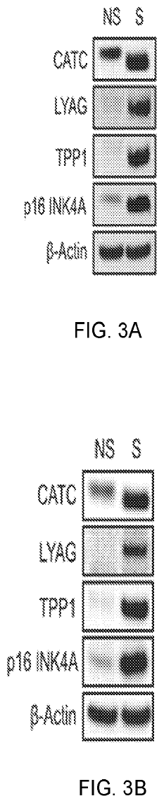

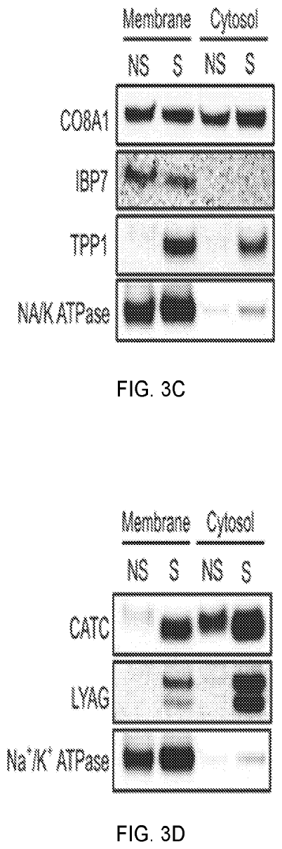

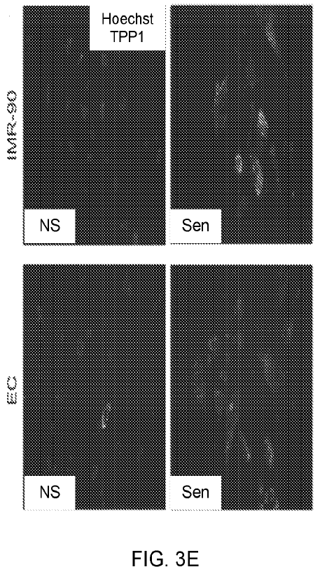

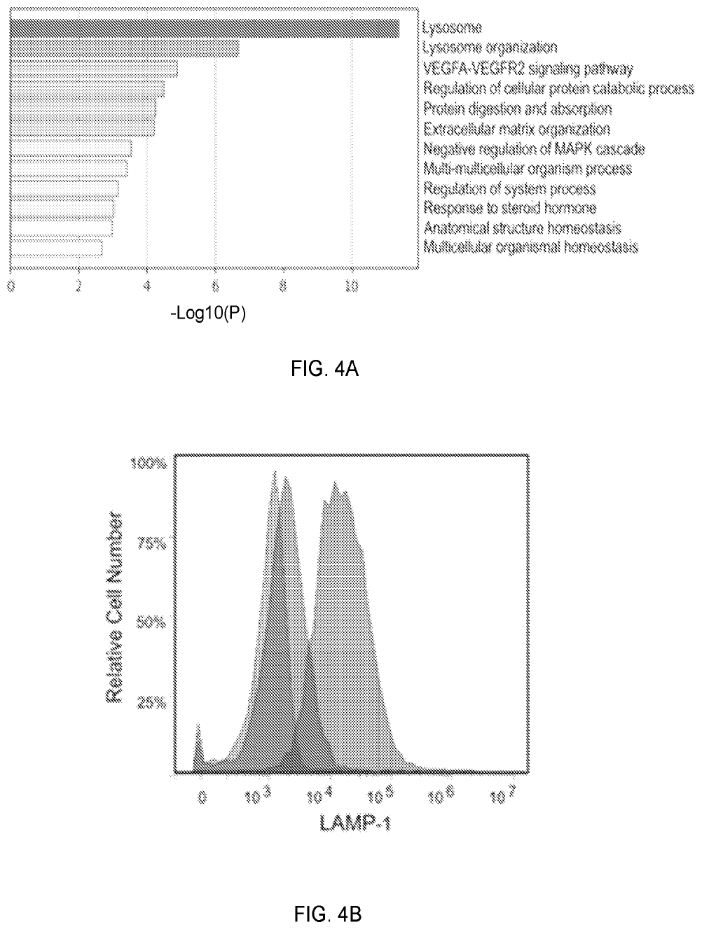

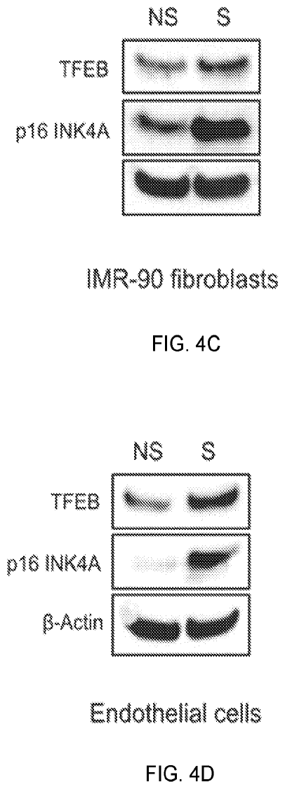

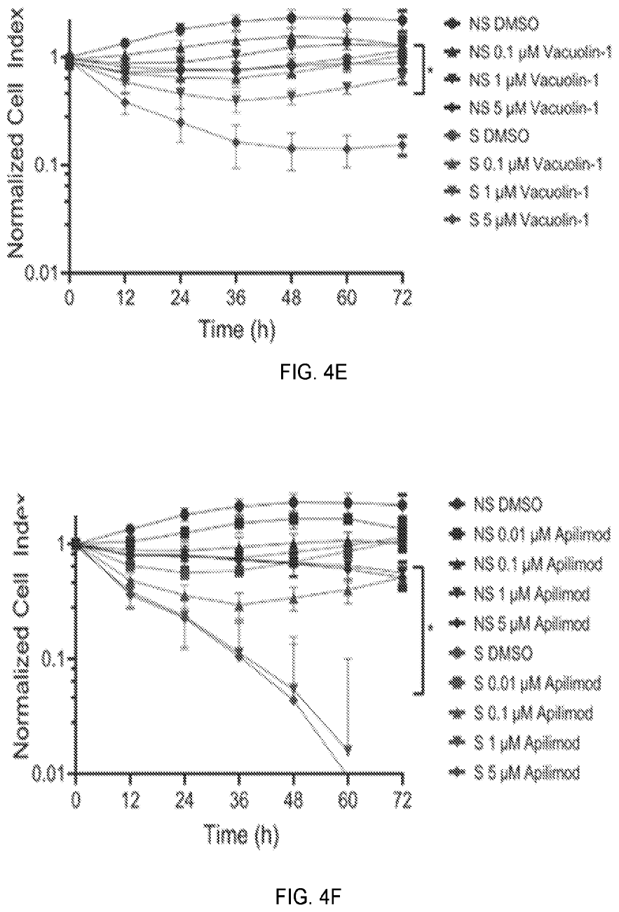

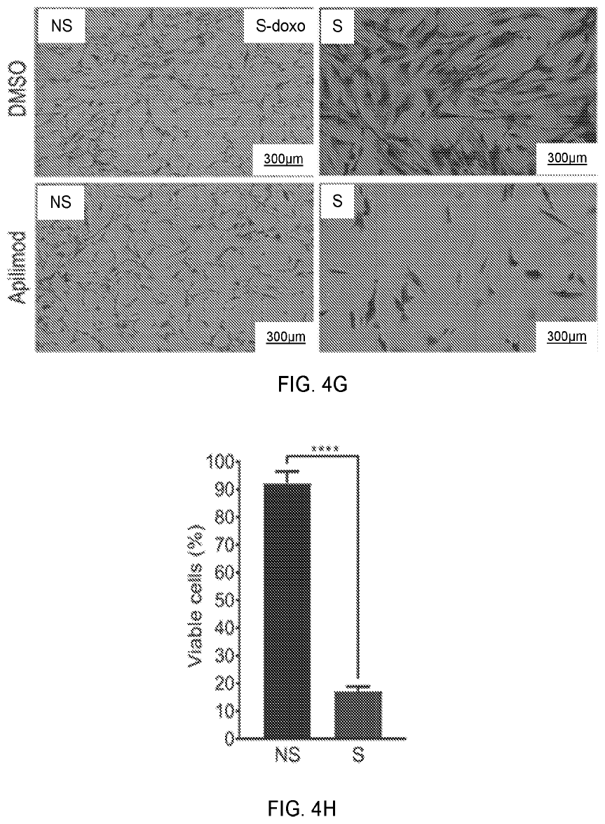

When evaluating the protein expression of these candidates, the increased protein expression of CATC, LYAG, and TPP1 was validated in the whole cell lysate of both senescent IMR-90 fibroblasts (FIG. 3A) and primary endothelial cells (FIG. 3B) compared to non-senescent (NS) control cells. Next, western blot analysis was performed on the membrane and cytosolic fractions of senescent(S) and non-senescent (NS) fibroblasts. Increased expression of CATC, LYAG and TPP1 in the membrane fraction of senescent cells compared to NS cells was observed. Na⁺/K⁺ ATPase was used to monitor cell fractionation (FIG. 3C). These results were further supported by immunofluorescence data that showed increased expression of TPP1 on the surface of senescent IMR-90 fibroblasts as well as senescent endothelial cells when compared to non-senescent controls (FIG. 3D).

### Example 3

### Senescent Cells Utilize Lysosomal Exocytosis to Survive

Analysis of 22 differentially expressed surface proteins common to both doxorubicin-treated (300 nM, 24 h) and irradiated (20 Gy) cells when compared to non-senescent (NS) control cells revealed that several lysosomal proteins are disproportionately abundant on the surface of senescent cells (FIG. 1A). The underlying mechanism by which senescent cells could present lysosomal proteins on their surface mechanism was further investigated as described below.

Given the abundance of lysosomal proteins presented on the surface of senescent cells, markers of lysosomal exocytosis were validated in senescent cells. Following lysosomal exocytosis, lysosomal membrane proteins such as LAMP-1 have been shown to present on the cell surface. Hence, surface expression of LAMP-1 is often used as a marker of lysosomal exocytosis. Flow cytometric analysis of senescent IMR-90 cells revealed a significant increase in LAMP-1 surface expression compared to non-senescent control cells (FIG. 1B). Transcription factor EB (TFEB) is known to be a master regulator of lysosomal biogenesis and autophagy. TFEB is also known to regulate lysosomal exocytosis. Western blot analysis of senescent IMR-90 and primary endothelial cells showed increased expression of TFEB compared to non-senescent control cells (FIG. 1C).

Senescent cells are known to employ several mechanisms to increase their survival. For example, increased expression of the pro-survival BCL-family proteins has been widely studied. In fact, BCL antagonism has been the basis of many senolytic drugs (see, e.g., Zhu, et al., Aging Cell. 2016 June; 15(3):428-35). Vacuolin-1 was used to determine if lysosomal exocytosis is a mechanism for senescent cell survival. Vacuolin-1 is a potent inhibitor of Phosphatidylinositol-3-phosphate 5-kinase type III (PIKfyve), which is a key player in lysosomal exocytosis allowing lysosomes to fuse with the plasma membrane. The viability assay was performed in senescent IMR-90 cells using xCELLigence Real-Time Cell Analysis (RTCA) system that uses label-free cellular impedance to monitor cell health and viability over time. Results demonstrated that blocking lysosomal exocytosis via vacuolin-1 selectively killed senescent IMR-90 cells in a dose-dependent manner, with a mean normalized cell index of 1.2 for NS cells and 0.4 for senescent cells after 48 hours of treatment with 1 μM of vacuolin-1 (FIG. 1D).

Apilimod is a clinical PIKfyve inhibitor. Apilimod was used to further validate the senolytic effects of PIKfyve inhibitors. The results demonstrated that, like vacuolin-1, apilimod caused dose-dependent declines in senescent cell viability. No senescent cells survived after 48 hours of treatment with 1 μM and 5 μM of apilimod (FIG. 1D). To further characterize the potent cytotoxic effect of apilimod on non-senescent versus senescent cells, IMR-90 fibroblasts were treated with 1 μM of apilimod. Light microscopy images of crystal violet-stained cells after 48 hours of apilimod treatment demonstrated a significant decrease in the viability of senescent cells, with no effect on non-senescent cell viability (FIG. 1E). Quantification of adherent cells revealed 92% non-senescent cell viability compared to only 17% viable cells in the senescent condition (FIG. 1E). Of note, the decrease in viable apilimod-treated non-senescent cells after 48 hours of treatment is likely in part due to a decrease in proliferation rather than viability of apilimod-treated cells.

### Example 4

### Validation of Senescent Cell Surface Markers by Flow Cytometry

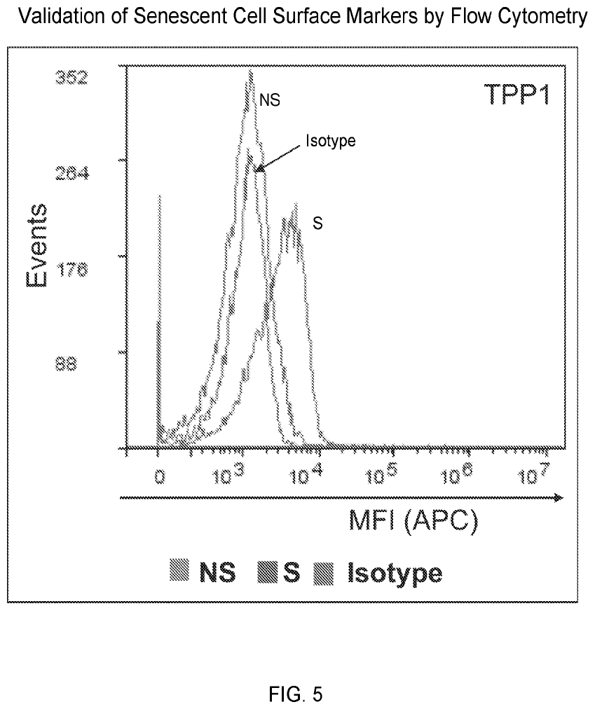

IMR90 cells were treated with Doxorubicin (DOX, 300 nM) for 10 days to induce senescence. Senescent cells (S-Doxo) and non-senescent cells (NS) were harvested and stained with the primary antibody and APC-labeled secondary antibody for 30 minutes at 4° C. The cells were analyzed by flow cytometry. The cells were stained with anti-TPP1 antibody (clone 2E12, EMD Millipore). The MFI of the isotype control (gray line) is 1154, the MFI of the non-senescent cells (blue line) is 1134 and the MFI of senescent cells (red line) is 3251. The data demonstrated that Lamp-1 and TPP1 were displayed on the surface of senescent cells. The results are shown in FIG. 5.

### Example 5

### Therapeutic Removal of Senescent Cells

In this example, a patient (55-year-old male) visits a physician's clinic and presents signs and symptoms of atherosclerosis and hypertension. A healthcare provider suspects that the targeted removal of senescent cells in the patient will improve the patient's cardiovascular ailments.

A tissue sample and blood sample demonstrate elevated levels of the following proteins: CO8A1, CATC, LYAG, IBP7, TPP1. These markers indicate the presence of senescent cells at higher levels that normal for a patient his age.

The patient is also administered a drug conjugate (intravenously) for killing senescent cells. The conjugate includes an antibody (that recognizes CO8A1) and a cytotoxic agent (i.e., a toxic peptide). The patient is also administered a senolytic agent. The patient's blood pressure gradually improves. The healthcare provider continues to monitor atherosclerosis and hypertension.

### Methods of Use

Embodiments include a method of diagnosing an ailment (i.e., a senescence-associated disease or disorder) or determining a prognosis of a test subject with the ailment. The method can include measuring expression levels of one or more biomarkers to identify those that have altered levels of expression. Embodiments also include therapies for treating a senescence-associated disease or disorder. In one embodiment, a method includes administering to a pharmaceutical formulation containing a therapeutic agent that selectively kills senescent cells (i.e., selectively kills senescent cells over non-senescent cells). A treatment regimen can include administering a pharmaceutical formulation for a time sufficient and in an amount sufficient to selectively kill senescent cells.

Compositions in accordance with embodiments described herein have desirable properties, such as desirable solubility, viscosity, syringeability and stability. Lyophilates in accordance with embodiments described herein have desirable properties, as well, such as desirable recovery, stability and reconstitution.

In an embodiment, the pH of the pharmaceutical formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9.

In an embodiment, the pH of the pharmaceutical formulation is from about 1 to about 14, about 2 to about 14, about 3 to about 14, about 4 to about 14, about 5 to about 14, about 6 to about 14, about 7 to about 14, about 8 to about 14, about 9 to about 14, about 10 to about 14, about 11 to about 14, about 12 to about 14, about 13 to about 14, about 1 to about 10, about 2 to about 10, about 3 to about 10, about 4 to about 10, about 5 to about 10, about 6 to about 10, about 7 to about 10, about 8 to about 10, about 9 to about 10, about 1 to about 11, about 2 to about 11, about 3 to about 11, about 4 to about 11, about 5 to about 11, about 6 to about 11, about 7 to about 11, about 8 to about 11, about 9 to about 11, about 10 to about 11, about 1 to about 12, about 2 to about 12, about 3 to about 12, about 4 to about 12, about 5 to about 12, about 6 to about 12, about 7 to about 12, about 8 to about 12, about 9 to about 12, about 10 to about 12, about 11 to about 12, about 1 to about 13, about 2 to about 13, about 3 to about 13, about 4 to about 13, about 5 to about 13, about 6 to about 13, about 7 to about 13, about 8 to about 13, about 9 to about 13, about 10 to about 13, about 11 to about 13, about 12 to about 13, about 1 to about 9, about 2 to about 9, about 3 to about 9, about 4 to about 9, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

TABLE 2
Proteins Upregulated with Doxo Treatment

| | Protein | Accession | Gene | Protein description | Log2(FC) | Protein |
|---|---|---|---|---|---|---|
| 1 | DYN2* | P50570 | DNM2 | Dynamin-2 | 6 | DYN2* |
| 2 | CO8A1 | P27658 | COL8A1 | Collagen alpha-1(VIII) chain | 4.7 | CO8A1 |
| 3 | CAH2 | P00918 | CA2 | Carbonic anhydrase 2 | 4.6 | CAH2 |
| 4 | COR1C | Q9ULV4 | CORO1C | Coronin-1C | 4.3 | COR1C |
| 5 | ADT2 | P05141 | SLC25A5 | ADPATP translocase 2 | 3.7 | ADT2 |
| 6 | UBA1 | P22314 | UBA1 | Ubiquitin-like modifier- | 3.6 | UBA1 |
| | | | | activating enzyme 1 | | |
| 7 | SYEP | P07814 | EPRS | Bifunctional glutamateproline-- | 3.4 | SYEP |
| | | | | tRNA ligase | | |
| 8 | VCAM1 | P19320 | VCAM1 | Vascular cell adhesion protein 1 | 3.3 | VCAM1 |
| 9 | CATC | P53634 | CTSC | Dipeptidyl peptidase 1 | 3.2 | CATC |
| 10 | DNJA2* | O60884 | DNAJA2 | DnaJ homolog subfamily A member 2 | 3.2 | DNJA2* |
| 11 | IF5A1 | P63241 | EIF5A | Eukaryotic translation | 3.2 | IF5A1 |
| | | | | initiation factor 5A-1 | | |
| 12 | HS90A | P07900 | HSP90AA1 | Heat shock protein HSP 90-alpha | 3.1 | HS90A |
| 13 | HS90B | P08238 | HSP90AB1 | Heat shock protein HSP 90-beta | 3.1 | HS90B |
| 14 | GARS | P41250 | GARS | Glycine--tRNA ligase | 3 | GARS |
| 15 | PGH2* | P35354 | PTGS2 | Prostaglandin GH synthase 2 | 3 | PGH2* |
| 16 | LYAG | P10253 | GAA | Lysosomal alpha-glucosidase | 2.6 | LYAG |
| 17 | PSA5 | P28066 | PSMA5 | Proteasome subunit alpha type-5 | 2.4 | PSA5 |
| 18 | TPIS | P60174 | TPI1 | Triosephosphate isomerase | 2.4 | TPIS |
| 19 | HYOU1 | Q9Y4L1 | HYOU1 | Hypoxia up-regulated protein 1 | 2.3 | HYOU1 |
| 20 | PTGIS | Q16647 | PTGIS | Prostacyclin synthase | 2.3 | PTGIS |
| 21 | TRFE* | P02787 | TF | Serotransferrin | 2.3 | TRFE* |
| 22 | BGAL | P16278 | GLB1 | Beta-galactosidase | 2.2 | BGAL |
| 23 | GDN | P07093 | SERPINE2 | Glia-derived nexin | 2.2 | GDN |
| 24 | IBP7 | Q16270 | IGFBP7 | Insulin-like growth | 2.2 | IBP7 |
| | | | | factor-binding protein 7 | | |
| 25 | PRS4* | P62191 | PSMC1 | 26S proteasome regulatory subunit 4 | 2.2 | PRS4* |
| 26 | TPP1 | O14773 | TPP1 | Tripeptidyl-peptidase 1 | 2.2 | TPP1 |
| 27 | CA2D1 | P54289 | CACNA2D1 | Voltage-dependent calcium channel | 2.1 | CA2D1 |
| | | | | subunit alpha-2delta-1 | | |
| 28 | ENOA | P06733 | ENO1 | Alpha-enolase | 2.1 | ENOA |
| 29 | MA2B1 | O00754 | MAN2B1 | Lysosomal alpha-mannosidase | 2.1 | MA2B1 |
| 30 | CAVN2 | O95810 | CAVIN2 | Caveolae-associated protein 2 | 2 | CAVN2 |
| 31 | FBLN1 | P23142 | FBLN1 | Fibulin-1 | 2 | FBLN1 |
| 32 | LYOX | P28300 | LOX | Protein-lysine 6-oxidase | 1.9 | LYOX |
| 33 | PCP | P42785 | PRCP | Lysosomal Pro-X carboxypeptidase | 1.8 | PCP |
| 34 | DPP4 | P27487 | DPP4 | Dipeptidyl peptidase 4 | 1.7 | DPP4 |
| 35 | GLCM | P04062 | GBA | Lysosomal acid glucosylceramidase | 1.6 | GLCM |
| 36 | HEXB | P07686 | HEXB | Beta-hexosaminidase subunit beta | 1.5 | HEXB |
| 37 | PCYOX | Q9UHG3 | PCYOX1 | Prenylcysteine oxidase 1 | 1.5 | PCYOX |
| 38 | GALNS | P34059 | GALNS | N-acetylgalactosamine-6-sulfatase | 1.3 | GALNS |

TABLE 3
Proteins Upregulated with Irradiation (S-IR) Treatment

| | Protein | Accession | Gene | Protein description | Log2(FC) |
|---|---|---|---|---|---|
| 1 | CO8A1 | P27658 | COL8A1 | Collagen alpha-1(VIII) chain | 3.7 |
| 2 | VCAM1 | P19320 | VCAM1 | Vascular cell adhesion protein 1 | 3.6 |
| 3 | CAVN2 | O95810 | CAVIN2 | Caveolae-associated protein 2 | 2.8 |
| 4 | CA2D1 | P54289 | CACNA2D1 | Voltage-dependent calcium | 2.6 |
| | | | | channel subunit alpha-2delta-1 | |
| 5 | CATC | P53634 | CTSC | Dipeptidyl peptidase 1 | 2.6 |
| 6 | FBLN1 | P23142 | FBLN1 | Fibulin-1 | 2.6 |
| 7 | IBP7 | Q16270 | IGFBP7 | Insulin-like growth factor-binding | 2.5 |
| | | | | protein 7 | |
| 8 | PTGIS | Q16647 | PTGIS | Prostacyclin synthase | 2.5 |
| 9 | LYAG | P10253 | GAA | Lysosomal alpha-glucosidase | 2.2 |
| 10 | ITA1 | P56199 | ITGA1 | Integrin alpha-1 | 2.1 |
| 11 | SYNPO | Q8N3V7 | SYNPO | Synaptopodin | 2 |
| 12 | MA2B1 | O00754 | MAN2B1 | Lysosomal alpha-mannosidase | 1.9 |
| 13 | CREL1 | Q96HD1 | CRELD1 | Cysteine-rich with EGF-like | 1.8 |
| | | | | domain protein 1 | |
| 14 | LYOX | P28300 | LOX | Protein-lysine 6-oxidase | 1.8 |
| 15 | GALNS | P34059 | GALNS | N-acetylgalactosamine-6-sulfatase | 1.7 |
| 16 | PCP | P42785 | PRCP | Lysosomal Pro-X | 1.7 |
| | | | | carboxypeptidase | |
| 17 | GOT1B | Q9Y3E0 | GOLT1B | Vesicle transport protein GOT1B | 1.6 |
| 18 | FKB11 | Q9NYL4 | FKBP11 | Peptidyl-prolyl cis-trans | 1.5 |
| | | | | isomerase FKBP11 | |
| 19 | DPP4 | P27487 | DPP4 | Dipeptidyl peptidase 4 | 1.4 |
| 20 | PCYOX | Q9UHG3 | PCYOX1 | Prenylcysteine oxidase 1 | 1.4 |
| 21 | TPP1 | O14773 | TPP1 | Tripeptidyl-peptidase 1 | 1.4 |
| 22 | HEXB | P07686 | HEXB | Beta-hexosaminidase subunit beta | 1.3 |
| 23 | GLCM | P04062 | GBA | Lysosomal acid | 1.2 |
| | | | | glucosylceramidase | |
| 24 | TOR1A | O14656 | TOR1A | Torsin-1A | 1.1 |

TABLE 4
Proteins Upregulated with both Doxo and Irradiation (S-IR) Treatment

| | Protein | Accession | Gene | Protein description | Log2(doxo) | Log2(IR) |
|---|---|---|---|---|---|---|
| 1 | CO8A1 | P27658 | COL8A1 | Collagen alpha-1(VIII) chain | 4.7 | 3.7 |
| 2 | VCAM1 | P19320 | VCAM1 | Vascular cell adhesion protein 1 | 3.3 | 3.6 |
| 3 | CATC | P53634 | CTSC | Dipeptidyl peptidase 1 | 3.2 | 2.6 |
| 4 | LYAG | P10253 | GAA | Lysosomal alpha-glucosidase | 2.6 | 2.2 |
| 5 | PTGIS | Q16647 | PTGIS | Prostacyclin synthase | 2.3 | 2.5 |
| 6 | IBP7 | Q16270 | IGFBP7 | Insulin-like growth factor- | 2.2 | 2.5 |
| | | | | binding protein 7 | | |
| 7 | TPP1 | O14773 | TPP1 | Tripeptidyl-peptidase 1 | 2.2 | 1.4 |
| 8 | CA2D1 | P54289 | CACNA2D1 | Voltage-dependent calcium channel | 2.1 | 2.6 |
| | | | | subunit alpha-2delta-1 | | |
| 9 | MA2B1 | O00754 | MAN2B1 | Lysosomal alpha-mannosidase | 2.1 | 1.9 |
| 10 | CAVN2 | O95810 | CAVIN2 | Caveolae-associated protein 2 | 2 | 2.8 |
| 11 | FBLN1 | P23142 | FBLN1 | Fibulin-1 | 2 | 2.6 |
| 12 | LYOX | P28300 | LOX | Protein-lysine 6-oxidase | 1.9 | 1.8 |
| 13 | PCP | P42785 | PRCP | Lysosomal Pro-X carboxypeptidase | 1.8 | 1.7 |
| 14 | DPP4 | P27487 | DPP4 | Dipeptidyl peptidase 4 | 1.7 | 1.4 |
| 15 | GLCM | P04062 | GBA | Lysosomal acid glucosylceramidase | 1.6 | 1.2 |
| 16 | HEXB | P07686 | HEXB | Beta-hexosaminidase subunit beta | 1.5 | 1.3 |
| 17 | PCYOX | Q9UHG3 | PCYOX1 | Prenylcysteine oxidase 1 | 1.5 | 1.4 |
| 18 | GALNS | P34059 | GALNS | N-acetylgalactosamine-6-sulfatase | 1.3 | 1.7 |

TABLE 5
Senescence Biomarkers from Doxo (S-Doxo) Treatment (adjusted p-value < 0.01)

| | | | | | Adj. p value |
|---|---|---|---|---|---|
| | Protein | Accession | Gene | Protein description | (Doxo/control) |
| 1 | TPP1 | O14773 | TPP1 | Tripeptidyl-peptidase 1 | 3.5E−03 |
| 2 | LYAG | P10253 | GAA | Lysosomal alpha-glucosidase | 3.5E−03 |
| 3 | VCAM1 | P19320 | VCAM1 | Vascular cell adhesion protein 1 | 3.5E−03 |
| 4 | GARS | P41250 | GARS | Glycine--tRNA ligase | 3.5E−03 |
| 5 | CATC | P53634 | CTSC | Dipeptidyl peptidase 1 | 3.5E−03 |
| 6 | NCEH1 | Q6PIU2 | NCEH1 | Neutral cholesterol ester hydrolase 1 | 3.5E−03 |
| 7 | CSPG4 | Q6UVK1 | CSPG4 | Chondroitin sulfate proteoglycan 4 | 3.5E−03 |
| 8 | ELOV1 | Q9BW60 | ELOVL1 | Elongation of very long chain fatty | 3.5E−03 |
| | | | | acids protein 1 | |
| 9 | CO8A1 | P27658 | COL8A1 | Collagen alpha-1(VIII) chain | 3.5E−03 |
| 10 | IBP7 | Q16270 | IGFBP7 | Insulin-like growth factor-binding | 3.5E−03 |
| | | | | protein 7 | |
| 11 | PTGIS | Q16647 | PTGIS | Prostacyclin synthase | 3.5E−03 |
| 12 | MA2B1 | O00754 | MAN2B1 | Lysosomal alpha-mannosidase | 4.0E−03 |
| 13 | CAH2 | P00918 | CA2 | Carbonic anhydrase 2 | 4.9E−03 |
| 14 | GLCM | P04062 | GBA | Lysosomal acid glucosylceramidase | 4.9E−03 |
| 15 | S10A6 | P06703 | S100A6 | Protein S100-A6 | 4.9E−03 |
| 16 | DPP4 | P27487 | DPP4 | Dipeptidyl peptidase 4 | 4.9E−03 |
| 17 | LYOX | P28300 | LOX | Protein-lysine 6-oxidase | 4.9E−03 |
| 18 | PCP | P42785 | PRCP | Lysosomal Pro-X carboxypeptidase | 4.9E−03 |
| 19 | HACD3 | Q9P035 | HACD3 | Very-long-chain | 4.9E−03 |
| | | | | (3R)-3-hydroxyacyl-CoA | |
| | | | | dehydratase 3 | |
| 20 | SDCB1 | O00560 | SDCBP | Syntenin-1 | 5.1E−03 |
| 21 | MGST3 | O14880 | MGST3 | Microsomal glutathione | 5.1E−03 |
| | | | | S-transferase 3 | |
| 22 | EZRI | P15311 | EZR | Ezrin | 5.1E−03 |
| 23 | LEG3 | P17931 | LGALS3 | Galectin-3 | 5.1E−03 |
| 24 | FBLN1 | P23142 | FBLN1 | Fibulin-1 | 5.1E−03 |
| 25 | PCYOX | Q9UHG3 | PCYOX1 | Prenylcysteine oxidase 1 | 5.1E−03 |
| 26 | HEXB | P07686 | HEXB | Beta-hexosaminidase subunit beta | 5.4E−03 |
| 27 | GALNS | P34059 | GALNS | N-acetylgalactosamine-6-sulfatase | 8.0E−03 |
| 28 | ESYT1 | Q9BSJ8 | ESYT1 | Extended synaptotagmin-1 | 8.0E−03 |
| 29 | ACTN4 | O43707 | ACTN4 | Alpha-actinin-4 | 8.2E−03 |
| 30 | DHCR7 | Q9UBM7 | DHCR7 | 7-dehydrocholesterol reductase | 9.2E−03 |

TABLE 6
Senescence Biomarkers from Irradiation (S-IR) Treatment (adjusted p-value < 0.01)

| | | | | | Adj. p value |
|---|---|---|---|---|---|
| | Protein | Accession | Gene | Protein description | (Irr/control) |
| 1 | LYAG | P10253 | GAA | Lysosomal alpha-glucosidase | 3.2E−03 |
| 2 | VCAM1 | P19320 | VCAM1 | Vascular cell adhesion protein 1 | 3.2E−03 |
| 3 | FBLN1 | P23142 | FBLN1 | Fibulin-1 | 3.2E−03 |
| 4 | GALNS | P34059 | GALNS | N-acetylgalactosamine-6-sulfatase | 3.2E−03 |
| 5 | CATC | P53634 | CTSC | Dipeptidyl peptidase 1 | 3.2E−03 |
| 6 | IBP7 | Q16270 | IGFBP7 | Insulin-like growth factor-binding | 3.2E−03 |
| | | | | protein 7 | |
| 7 | LRC15 | Q8TF66 | LRRC15 | Leucine-rich repeat-containing | 3.2E−03 |
| | | | | protein 15 | |
| 8 | CREL1 | Q96HD1 | CRELD1 | Cysteine-rich with EGF-like | 3.2E−03 |
| | | | | domain protein 1 | |
| 9 | EZRI | P15311 | EZR | Ezrin | 3.2E−03 |
| 10 | CAVN2 | O95810 | CAVIN2 | Caveolae-associated protein 2 | 3.5E−03 |
| 11 | NCEH1 | Q6PIU2 | NCEH1 | Neutral cholesterol ester hydrolase 1 | 3.5E−03 |
| 12 | PCYOX | Q9UHG3 | PCYOX1 | Prenylcysteine oxidase 1 | 3.5E−03 |
| 13 | SDCB1 | O00560 | SDCBP | Syntenin-1 | 3.5E−03 |
| 14 | MA2B1 | O00754 | MAN2B1 | Lysosomal alpha-mannosidase | 3.5E−03 |
| 15 | TPP1 | O14773 | TPP1 | Tripeptidyl-peptidase 1 | 3.5E−03 |
| 16 | HEXB | P07686 | HEXB | Beta-hexosaminidase subunit beta | 3.5E−03 |
| 17 | PCP | P42785 | PRCP | Lysosomal Pro-X carboxypeptidase | 3.5E−03 |
| 18 | PTGIS | Q16647 | PTGIS | Prostacyclin synthase | 3.5E−03 |
| 19 | CSPG4 | Q6UVK1 | CSPG4 | Chondroitin sulfate proteoglycan 4 | 3.5E−03 |
| 20 | SYNPO | Q8N3V7 | SYNPO | Synaptopodin | 3.5E−03 |
| 21 | CD248 | Q9HCU0 | CD248 | Endosialin | 3.5E−03 |
| 22 | FKB11 | Q9NYL4 | FKBP11 | Peptidyl-prolyl cis-trans isomerase | 3.5E−03 |
| | | | | FKBP11 | |
| 23 | GOT1B | Q9Y3E0 | GOLT1B | Vesicle transport protein GOT1B | 4.2E−03 |
| 24 | DPP4 | P27487 | DPP4 | Dipeptidyl peptidase 4 | 6.2E−03 |
| 25 | CO8A1 | P27658 | COL8A1 | Collagen alpha-1(VIII) chain | 7.4E−03 |
| 26 | ITA1 | P56199 | ITGA1 | Integrin alpha-1 | 7.4E−03 |
| 27 | LYOX | P28300 | LOX | Protein-lysine 6-oxidase | 7.5E−03 |
| 28 | GLCM | P04062 | GBA | Lysosomal acid glucosylceramidase | 9.4E−03 |
| 29 | TOR1A | O14656 | TOR1A | Torsin-1A | 9.7E−03 |
| 30 | PHB2 | Q99623 | PHB2 | Prohibitin-2 | 9.7E−03 |

1. A method of distinguishing non-senescent cells from senescent cells, the method comprising a step of comparing levels of one or more biomarkers from the non-senescent cells and senescent cells
wherein the biomarkers are comprised of proteins selected from LYAG, TPP1 and LAMP-1.

2. The method of claim 1, wherein the one or more biomarkers are further comprised of proteins selected from CO8A1, CATC, and IBP7.

3. The method of claim 1, wherein the one or more biomarkers is are further comprised of proteins selected from CO8A1, VCAM1, CATC, PTGIS, IBP7, CA2D1, MA2B1, CAVN2, FBLN1, LYOX, PCP, DPP4, GLCM, HEXB, PCYOX and GGALNS.

4. The method of claim 1, wherein the one or more biomarkers is are further comprised of proteins selected from VCAM1, GARS, CATC, NCEH1, CSPG4, ELOV1, CO8A1, IBP7, PTGIS, MA2B1, CAH2, GLCM, S10A6, DPP4, LYOX, PCP, HACD3, SDCB1, MGST3, EZRI, LEG3, FBLN1, PCYOX, HEXB, GALNS, ESYT1, ACTN4 and DHCR7.

5. The method of claim 1, wherein the one or more biomarkers are further comprised of proteins selected from VCAM1, FBLN1, GALNS, CATC, IBP7, LRC15, CREL1, EZRI, CAVN2, NCEH1, PCYOX, SDCB1, MA2B1, HEXB, PCP, PTGIS, CSPG4, SYNPO, CD248, FKB11, GOT1B, DPP4, CO8A1, ITA1, LYOX, GLCM, TOR1A and PHB2.

6. The method of claim 1, further comprising a step of selectively removing senescent cells from non-senescent cells.

7. The method of claim 1, further comprising a step of selectively labelling senescent cells.

8. A method of detecting an ailment or determining a prognosis of an ailment, the method comprising
a) detecting levels of one or more biomarkers in a sample from a test subject,
b) identifying the ailment or determining the prognosis of the ailment based on elevated levels of expression of the one or more biomarkers in the sample, and
c) treating the subject with a senolytic agent to prevent or ameliorate the ailment,
wherein the ailment is a senescence-associated disease or disorder,
wherein the biomarkers are comprised of proteins selected from LYAG, TPP1 and LAMP-1.

9. The method of claim 8, wherein the one or more biomarkers is are further comprised of proteins selected from CO8A1, CATC, and IBP7, TPP1.

10. The method of claim 1, wherein the one or more biomarkers is are further comprised proteins selected from CO8A1, VCAM1, CATC, PTGIS, IBP7, CA2D1, MA2B1, CAVN2, FBLN1, LYOX, PCP, DPP4, GLCM, HEXB, PCYOX and GGALNS.

11. The method of claim 8, wherein the one or more biomarkers is are further comprised of proteins selected from VCAM1, GARS, CATC, NCEH1, CSPG4, ELOV1, CO8A1, IBP7, PTGIS, MA2B1, CAH2, GLCM, S10A6, DPP4, LYOX, PCP, HACD3, SDCB1, MGST3, EZRI, LEG3, FBLN1, PCYOX, HEXB, GALNS, ESYT1, ACTN4 and DHCR7.

12. The method of claim 8, wherein the one or more biomarkers is are further comprised proteins selected from LYAG, VCAM1, FBLN1, GALNS, CATC, IBP7, LRC15, CREL1, EZRI, CAVN2, NCEH1, PCYOX, SDCB1, MA2B1, HEXB, PCP, PTGIS, CSPG4, SYNPO, CD248, FKB11, GOT1B, DPP4, CO8A1, ITA1, LYOX, GLCM, TOR1A and PHB2.

13. The method of claim 8, wherein the senolytic agent inhibits lysosomal exocytosis.

14. The method of claim 13, wherein the senescence-associated disease or disorder is one or more of atherosclerosis, osteoarthritis, osteoporosis, hypertension, arthritis, cataracts, cancer, Alzheimer's disease, chronic obstructive pulmonary disease (COPD), idiopathic pulmonary fibrosis, hair graying, sarcopenia, adiposity, neurogenesis, fibrosis and glaucoma.

15.-34. (canceled)

35. The method of claim 8, further comprising a step of assessing the efficacy of a senolytic agent by repeating steps a), b) and c) after administration of a therapeutic amount of the senolytic agent.

36. The method of claim 8, wherein the step of treating the subject comprises administering a drug conjugate to the subject,
wherein the drug conjugate comprises (a) a senescent cell targeting agent configured to target and bind to the at least one senescent cell biomarker and (b) a cytotoxic agent, which kills the bound senescent cell.

37. The method of claim 36, wherein the targeting agent is an antibody or an antigen binding fragment thereof, an aptamer, a plastic antibody or a small molecule.

38. The method of claim 36, wherein the cytotoxic agent is a senolytic agent, a radioisotope, a toxin or a toxic peptide.

39.-41. (canceled)

42. A method of slowing the aging process and/or reducing signs of aging in a subject, the method comprising administering a therapeutic amount of a senolytic agent to the subject, wherein the senolytic agent inhibits lysosomal exocytosis.

43. The method of claim 42, wherein the senolytic agent is vacuolin-1 or apilimod.

44.-54. (canceled)
